SEQUENCE LISTING (82 sequences)

SEQ ID NO: 1 - DNA, 6 bp, Artificial Sequence
  Description of Artificial Sequence restriction site BglII as present in DNA and/or RNA originating from any source or from any organism
agatct

SEQ ID NO: 2 - DNA, 6 bp, Artificial Sequence
  Description of Artificial Sequence restriction site MunI as present in DNA and/or RNA originating from any source or from any organism
caattg

SEQ ID NO: 3 - DNA, 19 bp, Artificial Sequence
  Description of Artificial Sequence adapter
cggactagagtacactgtc

SEQ ID NO: 4 - DNA, 21 bp, Artificial Sequence
  Description of Artificial Sequence adapter
gatcgacagtgtactctagtc

SEQ ID NO: 5 - DNA, 23 bp, Artificial Sequence
  Description of Artificial Sequence adapter
aattcccaagagctctccagtac

SEQ ID NO: 6 - DNA, 20 bp, Artificial Sequence
  Description of Artificial Sequence adapter
tagtactggagagctcttgg

SEQ ID NO: 7 - DNA, 17 bp, Artificial Sequence
  Description of Artificial Sequence primer Bgl II
gagtacactgtcgatct

SEQ ID NO: 8 - DNA, 17 bp, Artificial Sequence
  Description of Artificial Sequence primer Mun I
gagagctcttggaattg

SEQ ID NO: 9 - DNA, 6 bp, Artificial Sequence
  Description of Artificial Sequence restriction site BclI as present in DNA and/or RNA originating from any source or from any organism
tgatca

SEQ ID NO: 10 - DNA, 6 bp, Artificial Sequence
  Description of Artificial Sequence restriction site XhoII as present in DNA and/or RNA originating from any source or from any organism
rgatcy

SEQ ID NO: 11 - DNA, 6 bp, Artificial Sequence
  Description of Artificial Sequence restriction site EcoRI as present in DNA and/or RNA originating from any source or from any organism
gaattc

SEQ ID NO: 12 - DNA, 6 bp, Artificial Sequence
  Description of Artificial Sequence restriction site AcsI/ApoI as present in DNA and/or RNA originating from any source or from any organism
raatty

SEQ ID NO: 13 - DNA, 5 bp, Artificial Sequence
  Description of Artificial Sequence adapter ligation
gatct

SEQ ID NO: 14 - DNA, 5 bp, Artificial Sequence
  Description of Artificial Sequence adapter ligation
agatc

SEQ ID NO: 15 - DNA, 5 bp, Artificial Sequence
  Description of Artificial Sequence adapter ligation
gatca

SEQ ID NO: 16 - DNA, 5 bp, Artificial Sequence
  Description of Artificial Sequence adapter ligation
tgatc

SEQ ID NO: 17 - DNA, 5 bp, Artificial Sequence
  Description of Artificial Sequence adapter ligation
gatcy

SEQ ID NO: 18 - DNA, 5 bp, Artificial Sequence
  Description of Artificial Sequence adapter ligation
rgatc

SEQ ID NO: 19 - DNA, 5 bp, Artificial Sequence
  Description of Artificial Sequence adapter ligation
caatt

SEQ ID NO: 20 - DNA, 5 bp, Artificial Sequence
  Description of Artificial Sequence adapter ligation
aattg

SEQ ID NO: 21 - DNA, 5 bp, Artificial Sequence
  Description of Artificial Sequence adapter ligation
gaatt

SEQ ID NO: 22 - DNA, 5 bp, Artificial Sequence
  Description of Artificial Sequence adapter ligation
aattc

SEQ ID NO: 23 - DNA, 5 bp, Artificial Sequence
  Description of Artificial Sequence adapter ligation
raatt

SEQ ID NO: 24 - DNA, 5 bp, Artificial Sequence
  Description of Artificial Sequence adapter ligation
aattr

SEQ ID NO: 25 - DNA, 5 bp, Artificial Sequence
  Description of Artificial Sequence restriction site Hga I (5/10)as present in DNA and/or RNA originating from any source or from any organism
gacgc

SEQ ID NO: 26 - DNA, 6 bp, Artificial Sequence
  Description of Artificial Sequence restriction site Acc65 I as present in      DNA and/or RNA originating from any so
ggtacc

SEQ ID NO: 27 - DNA, 6 bp, Artificial Sequence
  Description of Artificial Sequence restriction site Acc I as present in DNA and/or RNA originating from any source or from any organism
gtcgac

SEQ ID NO: 28 - DNA, 6 bp, Artificial Sequence
  Description of Artificial Sequence restriction site BsaH I as present in DNA and/or RNA originating from any source or from any organism
grcgyc

SEQ ID NO: 29 - DNA, 4 bp, Artificial Sequence
  Description of Artificial Sequence restriction site Aci I as present in DNA and/or RNA originating from any source or from any organism
ccgc

SEQ ID NO: 30 - DNA, 6 bp, Artificial Sequence
  Description of Artificial Sequence restriction site BsaH I as present in DNA and/or RNA originating from any source or from any organism
grcgcc

SEQ ID NO: 31 - DNA, 6 bp, Artificial Sequence
  Description of Artificial Sequence restriction site Afl III as present in DNA and/or RNA originating from any source or from any organism
acgcgt

SEQ ID NO: 32 - DNA, 6 bp, Artificial Sequence
  Description of Artificial Sequence restriction site Afl III as present in DNA and/or RNA originating from any source or from any organism
acatgt

SEQ ID NO: 33 - DNA, 6 bp, Artificial Sequence
  Description of Artificial Sequence restriction site Age I as present in DNA and/or RNA originating from any source or from any organism
accggt

SEQ ID NO: 34 - DNA, 6 bp, Artificial Sequence
  Description of Artificial Sequence restriction site Ava I as present in DNA and/or RNA originating from any source or from any organism
cccggg

SEQ ID NO: 35 - DNA, 8 bp, Artificial Sequence
  Description of Artificial Sequence restriction site SgrA I as present in DNA and/or RNA originating from any source or from any organism
caccggtg

SEQ ID NO: 36 - DNA, 6 bp, Artificial Sequence
  Description of Artificial Sequence restriction site Apa I as present in DNA and/or RNA originating from any source or from any organism
gggccc

SEQ ID NO: 37 - DNA, 6 bp, Artificial Sequence
  Description of Artificial Sequence restriction site ApaL I as present in DNA and/or RNA originating from any source or from any organism
gtgcac

SEQ ID NO: 38 - DNA, 6 bp, Artificial Sequence
  Description of Artificial Sequence restriction site Sfc I as present in DNA and/or RNA originating from any source or from any organism
ctgcag

SEQ ID NO: 39 - DNA, 6 bp, Artificial Sequence
  Description of Artificial Sequence restriction site Apo I as present in DNA and/or RNA originating from any source or from any organism
aaatty

SEQ ID NO: 40 - DNA, 6 bp, Artificial Sequence
  Description of Artificial Sequence restriction site Apo I as present in DNA and/or RNA originating from any source or from any organism
gaatty

SEQ ID NO: 41 - DNA, 8 bp, Artificial Sequence
  Description of Artificial Sequence restriction site Asc I as present in DNA and/or RNA originating from any source or from any organism
ggcgcgcc

SEQ ID NO: 42 - DNA, 6 bp, Artificial Sequence
  Description of Artificial Sequence restriction site Ase I as present in DNA and/or RNA originating from any source or from any organism
attaat

SEQ ID NO: 43 - DNA, 6 bp, Artificial Sequence
  Description of Artificial Sequence restriction site BsrF I as present in DNA and/or RNA originating from any source or from any organism
rccggy

SEQ ID NO: 44 - DNA, 8 bp, Artificial Sequence
  Description of Artificial Sequence restriction site SgrA I as present in DNA and/or RNA originating from any source or from any organism
crccggyg

SEQ ID NO: 45 - DNA, 6 bp, Artificial Sequence
  Description of Artificial Sequence restriction site Ava I as present in DNA and/or RNA originating from any source or from any organism
ctcgag

SEQ ID NO: 46 - DNA, 5 bp, Artificial Sequence
  Description of Artificial Sequence restriction site Ava II as present in DNA and/or RNA originating from any source or from any organism
ggwcc

SEQ ID NO: 47 - DNA, 7 bp, Artificial Sequence
  Description of Artificial Sequence restriction site PpuM I as present in DNA and/or RNA originating from any source or from any organism
rggaccy

SEQ ID NO: 48 - DNA, 7 bp, Artificial Sequence
  Description of Artificial Sequence restriction site PpuM I as present in DNA and/or RNA originating from any source or from any organism
rggtccy

SEQ ID NO: 49 - DNA, 6 bp, Artificial Sequence
  Description of Artificial Sequence restriction site Avr II as present in DNA and/or RNA originating from any source or from any organism
cctagg

SEQ ID NO: 50 - DNA, 6 bp, Artificial Sequence
  Description of Artificial Sequence restriction site BamH I as present in DNA and/or RNA originating from any source or from any organism
ggatcc

SEQ ID NO: 51 - DNA, 6 bp, Artificial Sequence
  Description of Artificial Sequence restriction site Ban I as present in DNA and/or RNA originating from any source or from any organism
ggcgcc

SEQ ID NO: 52 - DNA, 6 bp, Artificial Sequence
  Description of Artificial Sequence restriction site Ban II as present in DNA and/or RNA originating from any source or from any organism
gagctc

SEQ ID NO: 53 - DNA, 4 bp, Artificial Sequence
  Description of Artificial Sequence restriction site Bfa I as present in DNA and/or RNA originating from any source or from any organism
ctag

SEQ ID NO: 54 - DNA, 6 bp, Artificial Sequence
  Description of Artificial Sequence restriction site BsaH I as present in DNA and/or RNA originating from any source or from any organism
gacgyc

SEQ ID NO: 55 - DNA, 6 bp, Artificial Sequence
  Description of Artificial Sequence restriction site BsaH I as present in DNA and/or RNA originating from any source or from any organism
ggcgyc

SEQ ID NO: 56 - DNA, 6 bp, Artificial Sequence
  Description of Artificial Sequence restriction site BsaW I as present in DNA and/or RNA originating from any source or from any organism
wccggw

SEQ ID NO: 57 - DNA, 6 bp, Artificial Sequence
  Description of Artificial Sequence restriction site BsiE I as present in DNA and/or RNA originating from any source or from any organism
cgatcg

SEQ ID NO: 58 - DNA, 6 bp, Artificial Sequence
  Description of Artificial Sequence restriction site BsiE I as present in DNA and/or RNA originating from any source or from any organism
cggccg

SEQ ID NO: 59 - DNA, 6 bp, Artificial Sequence
  Description of Artificial Sequence restriction site Bsp1286 I as present in DNA and/or RNA originating from any source or from any organism
gagcac

SEQ ID NO: 60 - DNA, 6 bp, Artificial Sequence
  Description of Artificial Sequence restriction site Bsp1286 I as present in DNA and/or RNA originating from any source or from any organism
gwgcwc

SEQ ID NO: 61 - DNA, 6 bp, Artificial Sequence
  Description of Artificial Sequence restriction site BspH I as present in DNA and/or RNA originating from any source or from any organism
tcatga

SEQ ID NO: 62 - DNA, 6 bp, Artificial Sequence
  Description of Artificial Sequence restriction site Sty I as present in DNA and/or RNA originating from any source or from any organism
ccatgg

SEQ ID NO: 63 - DNA, 6 bp, Artificial Sequence
  Description of Artificial Sequence restriction site BsrF I as present in DNA and/or RNA originating from any source or from any organism
accggy

SEQ ID NO: 64 - DNA, 6 bp, Artificial Sequence
  Description of Artificial Sequence restriction site BsrF I as present in DNA and/or RNA originating from any source or from any organism
gccggy

SEQ ID NO: 65 - DNA, 6 bp, Artificial Sequence
  Description of Artificial Sequence restriction site BssH II as present in      DNA and/or RNA origin ating from any so FEATURE
gcgcgc

SEQ ID NO: 66 - DNA, 6 bp, Artificial Sequence
  Description of Artificial Sequence restriction site BstY I as present in DNA and/or RNA originating from any source or from any organism
agatcy

SEQ ID NO: 67 - DNA, 6 bp, Artificial Sequence
  Description of Artificial Sequence restriction site BstY I as present in DNA and/or RNA originating from any source or from any organism
ggatcy

SEQ ID NO: 68 - DNA, 6 bp, Artificial Sequence
  Description of Artificial Sequence restriction site Eae I as present in DNA and/or RNA originating from any source or from any organism
yggccr

SEQ ID NO: 69 - DNA, 6 bp, Artificial Sequence
  Description of Artificial Sequence restriction site Eae I as present in DNA and/or RNA originating from any source or from any organism
cggccr

SEQ ID NO: 70 - DNA, 6 bp, Artificial Sequence
  Description of Artificial Sequence restriction site Eae I as present in DNA and/or RNA originating from any source or from any organism
tggccr

SEQ ID NO: 71 - DNA, 6 bp, Artificial Sequence
  Description of Artificial Sequence restriction site Apo I as present in DNA and/or RNA originating from any source or from any organism
rattty

SEQ ID NO: 72 - DNA, 6 bp, Artificial Sequence
  Description of Artificial Sequence restriction site Nsi I as present in DNA and/or RNA originating from any source or from any organism
atgcat

SEQ ID NO: 73 - DNA, 6 bp, Artificial Sequence
  Description of Artificial Sequence restriction site Nsp I as present in DNA and/or RNA originating from any source or from any organism
rcatgy

SEQ ID NO: 74 - DNA, 8 bp, Artificial Sequence
  Description of Artificial Sequence restriction site Pac I as present in DNA and/or RNA originating from any source or from any organism
ttaattaa

SEQ ID NO: 75 - DNA, 7 bp, Artificial Sequence
  Description of Artificial Sequence restriction site PpuM I as present in DNA and/or RNA originating from any source or from any organism
rggwccy

SEQ ID NO: 76 - DNA, 7 bp, Artificial Sequence
  Description of Artificial Sequence restriction site Rsr II as present in DNA and/or RNA originating from any source or from any organism
cggwccg

SEQ ID NO: 77 - DNA, 8 bp, Artificial Sequence
  Description of Artificial Sequence restriction site Sbf I as present in DNA and/or RNA originating from any source or from any organism
cctgcagg

SEQ ID NO: 78 - DNA, 6 bp, Artificial Sequence
  Description of Artificial Sequence restriction site Sph I as present in DNA and/or RNA originating from any source or from any organism
gcatgc

SEQ ID NO: 79 - DNA, 4 bp, Artificial Sequence
  Description of Artificial Sequence restriction site Tsp509 I as present in DNA and/or RNA originating from any source or from any organism
aatt

SEQ ID NO: 80 - DNA, 4 bp, Artificial Sequence
  Description of Artificial Sequence methylase site as present in DNA and/or RNA originating from any source or from any organism
gatc

SEQ ID NO: 81 - DNA, 22 bp, Artificial Sequence
  Description of Artificial Sequence adapter Mun I
aattccaagagctctccagtac

SEQ ID NO: 82 - DNA, 18 bp, Artificial Sequence
  Description of Artificial Sequence adapter Mun I
agtactggagagctcttg

(19) United States
(12) Patent Application Publication
(10) Pub. No.: US 2005/0208513 A1
(43) Pub. Date: Sep. 22, 2005

(54) Detection of polymorphisms

(76) Inventors: Edwin Chukwura Agbo, Lelystad (NL); Marinus Frederick Willem te Pas, Putten (NL); Marinus Adrianus Smits, Harderwijk (NL)

Correspondence Address: TRASK BRITT, P.O. BOX 2550, SALT LAKE CITY, UT, 84110, US

(21) Appl. No.: 10/959,638

(22) Filed: Oct. 5, 2004

Related U.S. Application Data
(63) Continuation of application No. PCT/NL03/00253, filed on Apr. 4, 2003.

(30) Foreign Application Priority Data
Apr. 5, 2002 (EP) 02076336.3

(51) Int. Cl. C12Q 1/68; C12P 19/34

(52) U.S. Cl. 435/6; 435/91.2

(57) ABSTRACT

The present invention provides methods for producing a nucleic acid fingerprint and methods for detecting sequence polymorphisms between one or more genomes. The methods include fragmenting a nucleic acid into nucleic acid fragments with ends that are compatible to ligation with at least one adapter, performing a ligation reaction between the compatible ends of the nucleic acid fragments and at least one adapter, amplifying the nucleic acid fragments by using at least one amplification primer, and generating a nucleic acid fingerprint from the amplified fragments. A method according to the present invention permits high-resolution fingerprinting while maintaining stringency in a PCR reaction. In another aspect, the invention also provides a kit for preparing nucleic acid fingerprints according to methods of the invention.

### CROSS-REFERENCE TO RELATED APPLICATIONS

This application is a continuation of International Application No. PCT/NL03/00253, filed Apr. 4, 2003, designating the United States of America, corresponding to International Publication No. WO 03/087409 (published in English on Oct. 23, 2003), the contents of the entirety of which are incorporated by this reference.

### TECHNICAL FIELD

The invention relates to biotechnology in the field of molecular biology and more in particular, to genetic diversity analysis. More particularly, the invention relates to the genetic analysis of nucleotide sequence polymorphisms in DNA by nucleic acid fingerprinting.

### BACKGROUND

In order to assess genetic diversity among prokaryotic or eukaryotic organisms and to address questions regarding population structure and genetic relatedness, a variety of methods, predominantly genome fingerprinting methods, has been developed in recent years (see Mueller and Wolfenbarger for review).

Methods such as genome fingerprinting have been developed with the aim of providing an estimate, preferably essentially unbiased, of the total genome variance in a population of organisms, while minimizing errors due to sampling variance.

The amplified fragment length polymorphism (AFLP) method (Vos et al. 1995), for example, has shown great value for interspecies genotyping, but has been reported to offer low resolution in the typing of, for example, trypanosome subspecies (Agbo et al., 2002) and closely related strains in other taxa (Lindstedt et al., 2000).

AFLP is a DNA fingerprinting method that detects defined DNA restriction fragments by means of PCR amplification. The method comprises the steps of: a) digesting DNA with restriction endonuclease enzymes, b) ligating double-stranded (ds) adapters to the ends of the restriction fragments to which PCR primers can be annealed, c) amplifying a subset of the restriction fragments using two primers essentially complementary to the adapter, d) separating the amplified restriction fragments on denaturing polyacrylamide gels by gel-electrophoresis, and e) visualizing the separated restriction fragments by means of autoradiography, phospho-imaging, or other methods, thus obtaining a DNA fingerprint. Essentially, AFLP accesses subsets of polymorphisms at one or two restriction sites.

For most living organisms, except the smaller viruses, restriction digestion of the genomic DNA, or nucleic acid such as cDNA derived therefrom, generally results in such a large number of fragments that full separation into discrete fragments on a gel is no longer feasible. Therefore, often only a small fraction of the DNA fragments are selected to be detected. This selection, for example, occurs during PCR amplification or sometimes even haphazardly during visualization or detection on, for example, a gel. In case the selection occurs during PCR amplification, this is usually done by pre-selecting a subset of tagged restriction fragments, which pre-selection resides in the specific design of the oligonucleotides that are used as primers in the PCR amplification reaction, so that only a relatively small number of tagged restriction fragments will be amplified during the PCR amplification reaction.

As mentioned, in many applications, such as the typing of species, conventional DNA fingerprinting methods provide sufficient sensitivity to detect differences within otherwise identical DNA fingerprints, also known as DNA polymorphisms. Polymorphic positions in the DNA can be a suitable basis for the development of DNA markers.

However, in the case of comparative analysis of genomes of even more closely related organisms, wherein relatively little genetic variation is expected, such as the genetic variation within a subspecies, the genetic variation cannot always be revealed by using conventional genetic diversity analysis techniques. In such cases, methods capable of providing increased resolution power are needed in order to reveal nucleotide polymorphisms.

In order to accomplish such increased discriminatory power, it is desirable to achieve an even greater resolution of identifiable polymorphic fragments, i.e., to generate more fragments that potentially contain polymorphisms and to detect these in a robust way.

Conventional fingerprinting methods are generally able to detect polymorphisms in the target sites of the restriction endonucleases applied in the DNA digestion reaction, as well as polymorphisms in the adjacent nucleotide sequences, by virtue of the selective design of the PCR primers, but are unable to detect polymorphisms in other regions of the DNA fragments produced.

Several attempts have been made to generate more fragments that potentially contain polymorphisms and to detect these, for example, by using more restriction enzymes in one reaction for DNA digestion. Simons and co-workers (Simons et al., 1997) pioneered early attempts at the use of additional endonucleases in AFLP to gain access to more independent restriction sites in order to increase the discriminatory power of the method, but reported only slight improvement in fingerprint patterns compared to the standard procedure in which two restriction endonucleases are used.

Lindstedt and co-workers (Lindstedt et al., 2000) digested DNA with a set of three restriction endonuclease enzymes followed by adapter ligation and subsequent amplification by using a set of three primers. This method inherently compromises the stringency of the PCR amplification reaction, which may lead to variable results, and compromises the robustness of the method.

More recently, the use of three endonuclease enzymes in a genome fingerprinting method (Van der Wurff et al., 2000) was even reported to be very suitable for providing a lower number of potentially amplifiable fragments, thus providing a method for studying relatedness between more distantly related taxa.

In certain instances, such as in the case of genetic analysis of organisms to subspecies level, additional discriminatory power is required. For example, within parasites such as trypanosomes, these problems are often exemplified.

Trypanosomes are unicellular, protozoan parasites that cause debilitating disease (collectively called trypanosomosis) (Kassai, 1988) in man and animals. The disease is often fatal to man and animal. Trypanosoma brucei consists of three subspecies, which are indistinguishable by conventional morphological, biochemical and antigenic criteria, but differ in their geographical distribution and host specificity (Gibson et al., 1980).

Trypanosoma b. brucei is one of the trypanosomes that cause “nagana” in cattle, but does not cause disease in humans, because this subspecies is lysed by normal human serum. T. b. rhodesiense, and T. b. gambiense are the cause of human sleeping sickness in east and central West Africa, respectively. Trypanosoma b. gambiense and T. b. rhodesiense appear to differ in the mechanism of serum resistance (Hawking, 1973; Hawking, 1977). Agbo and co-workers have reported limited associations between different genetic marker loci and resistance in molecular signatures in human- and nonhuman-infective T. brucei isolates (Agbo et al., 2001). These and other studies have shown an urgent need for additional discriminatory power in fingerprinting techniques. Furthermore, it is now well established that T. b. rhodesiense and T. b. brucei genomes are more polymorphic than those of T. b. gambiense (Hide, 1999; Agbo et al., 2002). However, knowledge of the extent of genetic diversity of local parasite populations and the role of this diversity in the interactions of the parasite with both the animal and human hosts in related and distant populations remain poorly understood. The epidemiological analysis undertaken on stocks isolated during a human sleeping sickness epidemic in the Tororo district of Uganda (Hide et al., 1994), as well as other studies (MacLeod et al., 1999; MacLeod et al., 2001a; 2001b), provided unique opportunities to address these issues because a number of parameters that may provide insight into population structure of natural populations of trypanosomes were investigated. However, to use these kinds of opportunities, fingerprint techniques with additional discriminatory power are urgently needed.

### SUMMARY OF THE INVENTION

The invention provides a method wherein a nucleic acid may now be fragmented by any suitable process of nucleic acid fragmentation and wherein nucleic acid fragments are provided that are selectively ligatable to a pair of adapters such that a specific amplification of the nucleic acid fragments is allowed and wherein a nucleic acid fingerprint is generated from the amplified nucleic acid fragments.

The present inventors have now surprisingly found that polymorphisms, preferably in complex and closely related subspecies, may be detected by selecting specific adapter pairs, making the method suitable in a method that fragments nucleic acid in an unspecific manner. The invention provides a method that allows for the use of highly degenerative nucleic acid fragmentation methods in nucleic acid fingerprinting techniques.

The present invention provides a method for producing a nucleic acid fingerprint and comprises the step of providing, from a starting nucleic acid, a plurality of adapter ligatable nucleic acid fragments with ends that are compatible to one adapter, and wherein the nucleic acid fragments are obtained by fragmentation of the starting nucleic acid.

Of course, for fragmentation, a random fragmentation or, alternatively, a specific fragmentation, may be used. Random fragmentation may comprise the physical shearing of nucleic acids, while specific fragmentation may comprise such methods as enzymatic digestion. In a preferred embodiment, in order to get the most defined fragments, an endonuclease enzyme is used in a specific fragmentation procedure.

A method for producing a nucleic acid fingerprint according to the invention further comprises the step of performing a ligation reaction between the ends of the nucleic acid fragments and at least one adapter to produce, for example, adapter-ligated nucleic acid fragments. It is the specific choice of the adapters that allows for the use of any method of fragmentation of the nucleic acid in a method according to the present invention.

As long as the adapters allow for the performance of a next step in a method for producing a nucleic acid fingerprint according to the invention, which comprises amplifying the adapter-ligated nucleic acid fragments, by using at least one amplification primer essentially complementary to the nucleotide sequence of the at least one adapter. Such an amplification reaction may comprise any suitable DNA amplification reaction known to the person skilled in the art, such as a PCR amplification reaction.

Finally, a method for producing a nucleic acid fingerprint according to the invention comprises the step of generating from the amplified adapter-ligated nucleic acid fragments a nucleic acid fingerprint.

A method according to the invention comprising the random fragmentation of starting nucleic acid is particularly useful for analyzing DNA of parasites, which often contains very substantial amounts (often >80%) of A+T-rich, non-coding sequences, mainly consisting of repetitive sequences of micro-satellite DNA. On the basis of this method, a pair of adapters can now readily be designed for robust amplification of fragments generated.

A method according to the invention accesses additional independent restriction sites per analysis in comparison to the methods of the prior art. A method according to the invention permits, for example, the simultaneous use of three, four or more endonucleases, thereby providing greater resolution of identifiable polymorphic fragments, in combination with only one pair of adapters and cognate primers to maintain stringency in PCR amplification.

In one embodiment, a method according to the invention makes use of the fact that some endonucleases create cohesive ends that are similar to the overhanging or recessive ends created by other endonucleases. As a result, both restriction sites are compatible (in the sense of Watson-Crick base pairing) with one single adapter because they originate from different restriction site sequences. Therefore, a single adapter is ligated to the overhanging or recessive ends created by two different restriction enzymes.

In another embodiment according to the invention, the DNA is fragmented using unspecific methods for fragmentation and ligated to two adapters followed by selective amplification of DNA fragments.

A method of the invention may also comprise the cleavage or cutting of DNA into fragments by using specific methods of fragmentation such as an S1 nuclease digestion, which may specifically digest A+T-rich DNA sequences of non-coding regions of a genome. As long as the selective adapters allow for the specific amplification of the nucleic acid fragments, any fragmentation method is feasible according to the invention.

In a preferred embodiment of the present invention, the DNA is fragmented using specific methods for fragmentation such as by using restriction enzymes to digest the DNA to be studied into a large number of restriction fragments. The use of, for example, a set of four restriction enzymes provides discrimination at extra sites within the DNA, while only one pair of adapters and cognate primers is required as a direct result of which stringency in a PCR amplification reaction may be maintained.

Such embodiments of the present invention provides enhanced discriminatory power over conventional fingerprinting methods in the analysis of, for example, genetic differences among closely related parasite isolates, such as in trypanosomes.

A method according to the invention may comprise the use of two, three, four or even more restriction endonuclease enzymes, thereby allowing for the generation of varying numbers of identifiable polymorphic fragments, while at the same time, allowing for the use of only one pair of adapters and corresponding amplification primers, thereby maintaining the possibility to provide robust PCR reaction conditions.

By using a method according to the present invention, the inventors have been able to characterize a large number of trypanosome isolates to the subspecies level, and demonstrated the greater fingerprinting power of this approach over a well-established conventional method by the detection of additional DNA polymorphisms.

A method according to the invention may also be useful for identifying high-volume co-dominant genetic markers or differences in complex or closely related genomes and can easily be applied for genomic characterization of a variety of taxa.

### DESCRIPTION OF THE DRAWINGS

FIG. 1 shows a schematic illustration of one embodiment of a method according to the present invention in which four endonucleases and two adapters are used.

In step I, an amount of DNA is provided which is digested with four different restriction enzymes in step II. In this embodiment, a set of two pairs of restriction enzymes is used (BglII-BclI and EcoRI-MunI). The digestion generates 16 different restriction products in step III, corresponding to ten analogous sets of restriction fragments.

The solid lines represent restriction enzyme fragments of DNA. The boxes at the end of the various DNA fragments represent the respective restriction ends. Because, for each cognate pair of endonucleases, the cohesive ends created by one enzyme are compatible to the cohesive ends created by the other enzyme, the two adapters (BglII and MunI) also ligate to cohesive ends created by the endonuclease enzymes BclI and EcoRI, respectively.

Following the ligation reaction, five different groups of products arise (step IV): sets carrying BglII or MunI adapters at both ends (A, B); sets with BglII and MunI adapters at the ends of each fragment (heterosite) (C); sets with BglII or MunI adapter at only one end of fragment (D), and, sets that did not ligate to any adapter (E).

For selective PCR amplification and detection of fragments, MunI primer is fluorescently labeled. Note that irrespective of which of the two cognate primers is labeled, the same subset of representation products, comprising the heterosite fragments, is exponentially amplified and detected (IV, C).

FIG. 2 demonstrates the higher resolution power of the four endonuclease/two adapters approach.

Fingerprints resulting from intra- and inter-species analyses of trypanosome isolates demonstrate greater resolution of the four-endonuclease approach over the two-endonuclease approach. A, C and E represent dendograms from the samples analyzed with the four-endonuclease approach (using BglII-BclI/EcoRI-MunI). These are directly compared with profiles derived from samples processed with the two endonucleases (BglII and EcoRI), viz B, D and F, respectively.

A pair of adapters and primers (BglII and MunI) was used, respectively, to ligate and PCR amplify the resulting digestion products. The total number of bands from each tested sample is indicated.

FIG. 3 shows a dendogram from four-endonuclease analysis of 28 Trypanosoma brucei populations isolated during endemic period (1990-1992), based on similarity relationships, according to composite genomic patterns. Cluster A corresponded with T. b. brucei populations, whereas Clusters B and C each consisted of T. b. brucei and T. b. rhodesiense populations.

FIG. 4 shows the relationship between 28 T. brucei stocks or clones derived during endemic period survey between 1990-1992, inferred by numerical analysis of fingerprint data generated by six-endonuclease analysis. Subspecies identification “B” and “R” represent T. b. brucei and T. b. rhodesiense, respectively.

FIG. 5 shows an assessment of relationship between stocks isolated during endemic and epidemic periods in Busoga focus, Uganda. The extent relatedness of T. b. brucei (B) and T. b. rhodesiense (R) from epidemic (epi) and endemic (end) periods are grouped in Windows I, II and III.

FIG. 6 discloses a dendogram based on fingerprints from four-endonuclease analysis of T. b. brucei (B) and T. b. rhodesiense (R) populations isolated during endemic and epidemic periods in disparate geographical regions.

FIG. 7 shows fingerprint types and relationship between T. b. brucei (B), T. b. rhodesiense (R) and T. b. gambiense (G) populations from epidemic (epid) or endemic (end) periods in Busoga focus and other disparate locations. The two main clusters P and Q are further sub-clustered.

### DETAILED DESCRIPTION OF THE INVENTION

A method according to the invention provides a robust and reproducible technique for generating high numbers of amplifiable fragments from DNA and is, for example, used to detect high numbers of polymorphisms between, e.g., genomes.

When, for example, a set of four 6 bp-cutting endonucleases, each of which recognizes a different DNA sequence, is used during a genomic DNA digestion according to a method of the present invention, this set will simultaneously access independent sites. Further, according to the present invention, the set of four 6 bp-cutting endonucleases are preferably chosen such that they form paired sets, wherein each paired set comprises at least two restriction enzymes that recognize different DNA signatures but that create compatible cohesive ends, such compatible cohesive ends being ligatable to a single adapter. Therefore, when using a set of four 6 bp-cutting endonucleases, for example, chosen such that they form paired sets according to the invention, only two adapters are required to produce amplifiable fragments.

When, for example, a combination of four restriction enzymes is used that consist of a paired set of the restriction enzymes BglII and BclI and another paired set of the restriction enzymes EcoRI and MunI, only one pair of adapters, for example, a pair consisting of a BglII adapter and a MunI adapter (see Table 1), may be ligated to the various restriction fragments formed, thereby allowing the amplification of the restriction fragments by a single pair of cognate primers in PCR.

TABLE 1

A complementary set of adapters and PCR primers

| | | |
|---|---|---|
| Endonuclease | Adapter | Primer core sequence |

| | | |
|---|---|---|
| BglII | 5′-CGGACTAGAGTACACTGTC (SEQ ID NO:3) | 5′-6-FAM-GAGTACACTGTCGATCT |
| (A/GATCT) | 3′-CTGATCTCATGTGACAGCTAG (SEQ ID NO:4) | (SEQ ID NO:7) |
| (SEQ ID NO:1) | | |
| MunI | 5′-AATTCCCAAGAGCTCTCCAGTAC (SEQ ID NO:5) | 5′-6-FAM-GAGAGCTCTTGGAATTG |
| (C/AATTG) | 3′-GGTTCTCGAGAGGTCATGAT (SEQ ID NO:6) | (SEQ ID NO:8) |
| (SEQ ID NO:2) | | |

In the above embodiment according to the present invention, a BglII adapter is ligated to the cohesive ends created by the restriction enzymes BglII and BclI, while a MunI adapter is ligated to the cohesive ends created by the restriction enzymes MunI and EcoRI (See Table 2).

TABLE 2
Schematic representation of restriction site compatibility in relation to adapter ligation.

| Endonuclease | Restriction site | Sequence ID No. | Overhang ends formed | Adapter ligation | Sequence ID No. |
|---|---|---|---|---|---|
| BglII | 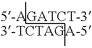 | (SEQ ID NO: 1) (SEQ ID NO: 1) | 5′-A GATCT-3′3′-TCTAG a-5′ | 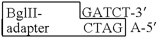 | (SEQ ID NO: 13) (SEQ ID NO: 14) |
| BclI | 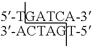 | (SEQ ID NO: 9) (SEQ ID NO: 9) | 5′-T GATCA-3′3′-ACTAG T-5′ | 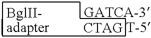 | (SEQ ID NO: 15) (SEQ ID NO: 16) |
| XhoII | 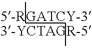 | (SEQ ID NO: 10) (SEQ ID NO: 10) | 5′-R GATCY-3′3′-YCTAG R-5′ | 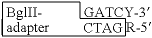 | (SEQ ID NO: 17) (SEQ ID NO: 18) |
| MunI | 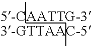 | (SEQ ID NO: 2) (SEQ ID NO: 2) | 5′-C AATTG-3′3′-GTTAA C-5′ | 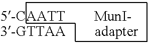 | (SEQ ID NO: 19) (SEQ ID NO: 20) |
| EcoRI | 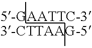 | (SEQ ID NO: 11) (SEQ ID NO: 11) | 5′-G AATTC-3′3′-CTTAA G-5′ | 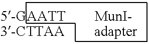 | (SEQ ID NO: 21) (SEQ ID NO: 22) |
| AcsI/ApoI | 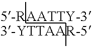 | (SEQ ID NO: 12) (SEQ ID NO: 12) | 5′-R AATTY-3′3′-YTTAA R-5′ | 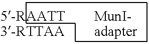 | (SEQ ID NO: 23) (SEQ ID NO: 24) |

The possibility of using a short adapter ligation step allows for a substantial reduction in the amount of time required to prepare DNA fingerprints according to a method of the invention.

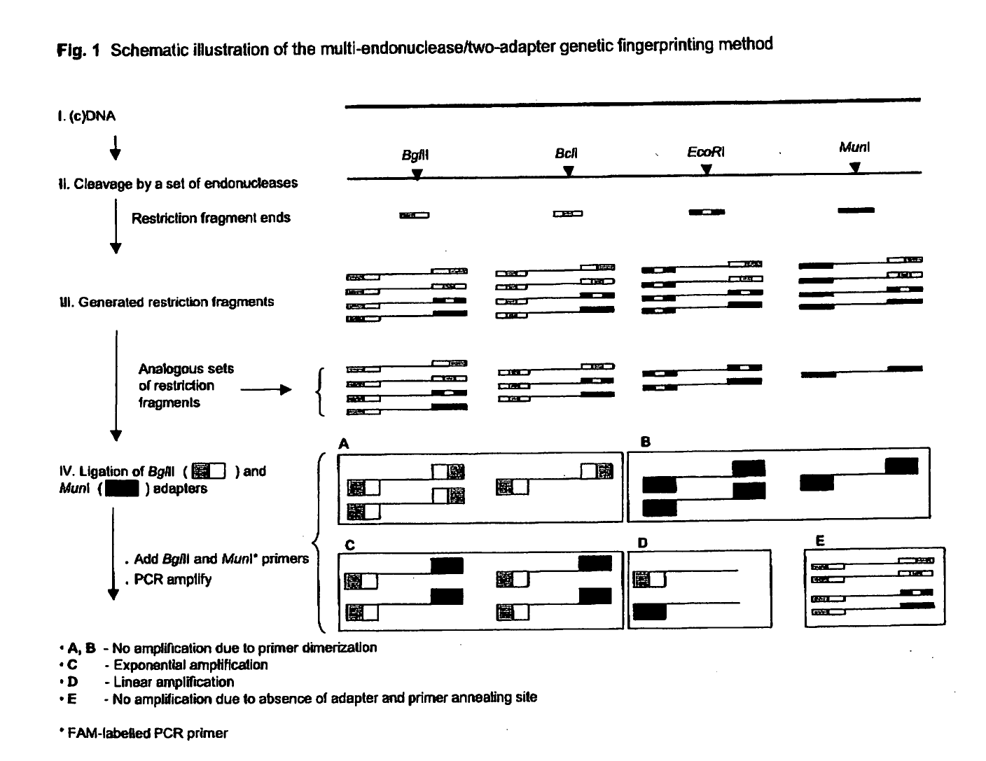

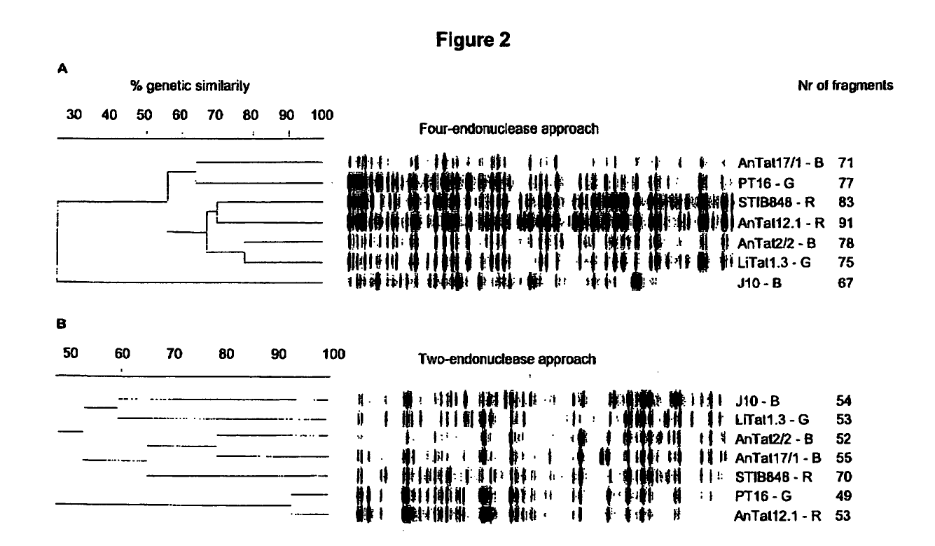

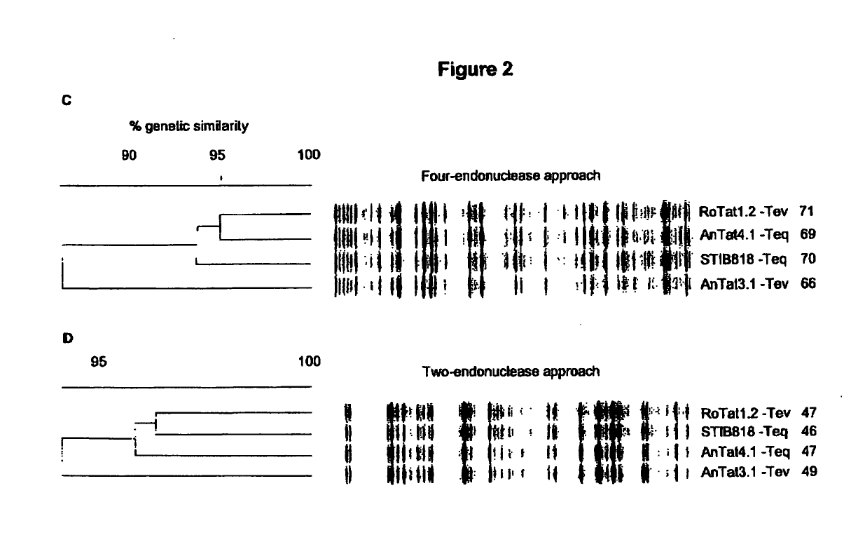

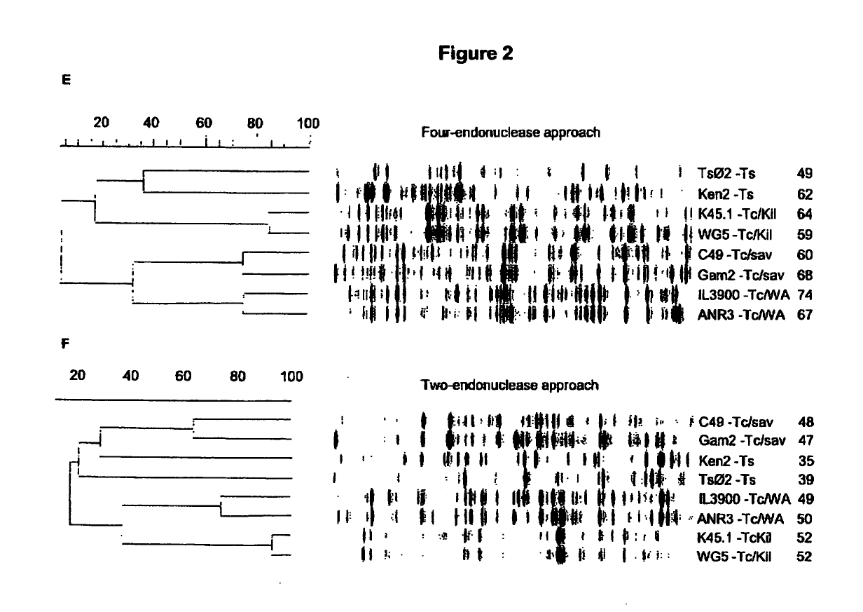

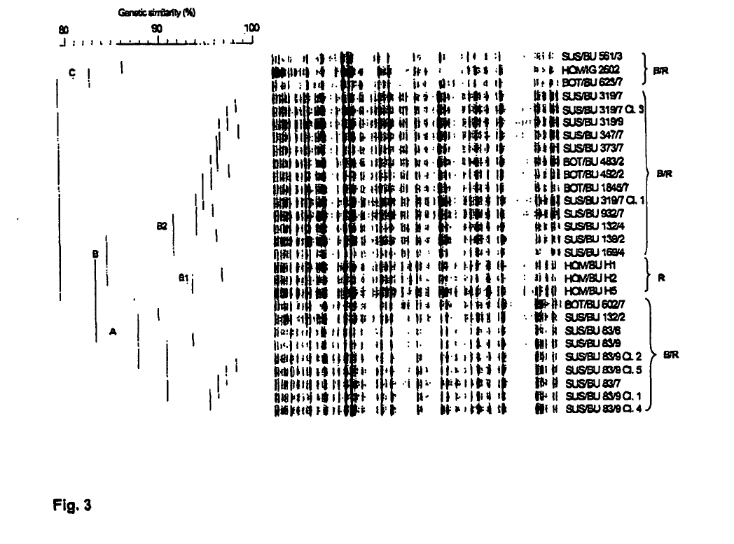

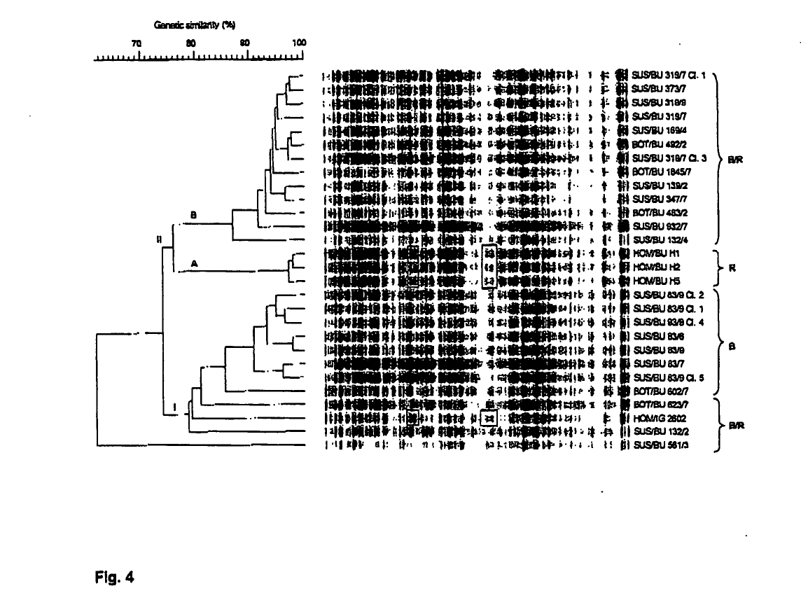

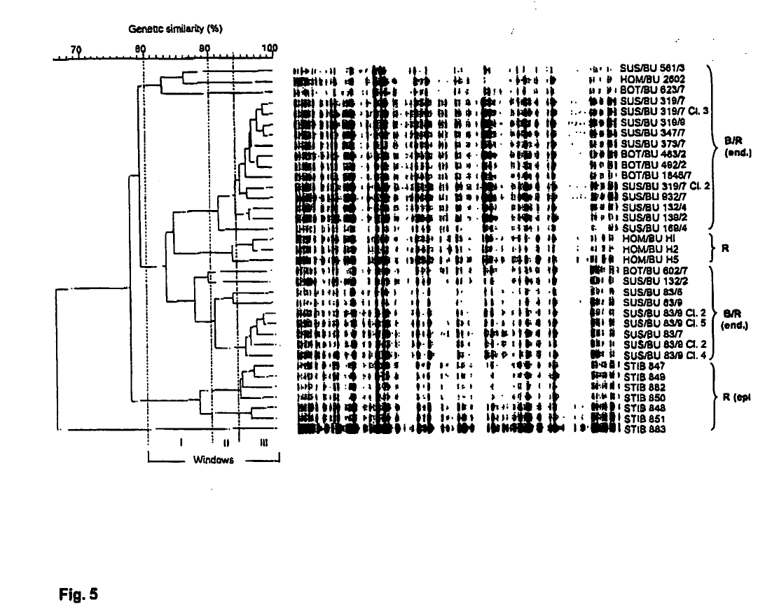

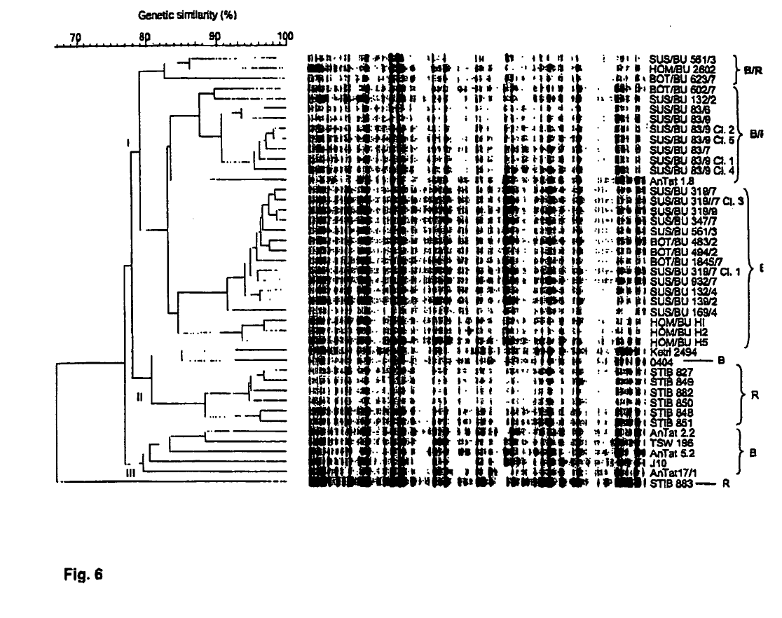

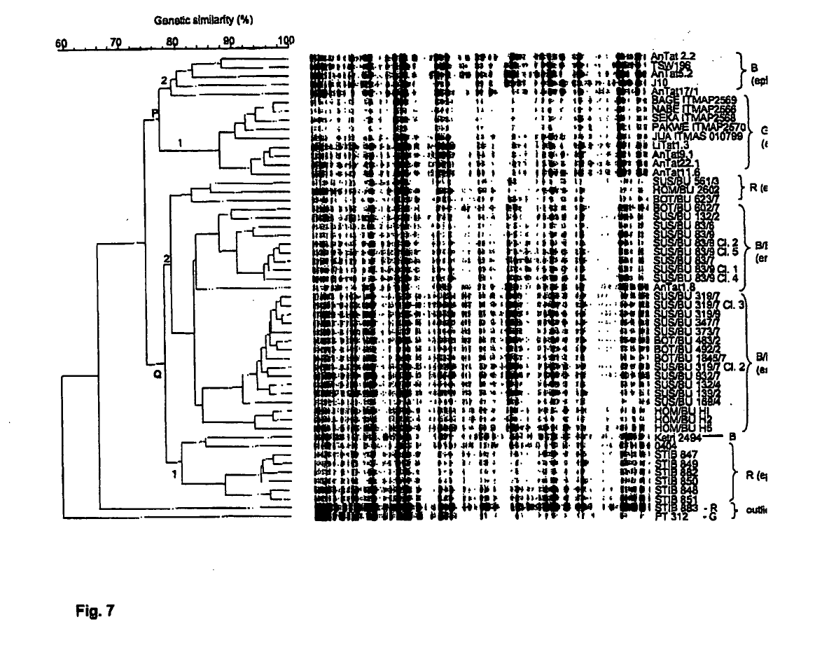

The above-described embodiment, wherein the restriction enzymes BglII, BclI, EcoRI and MunI are used in combination with adapter ligation to a BglII and a MunI adapter, results in the generation of five groups of reaction products (FIG. 1, Panels A-E). These consist of sets of fragments with (i) BglII or MunI adapters at both ends (homosite fragments) (A, B), (ii) BglII and MunI adapters at the opposite ends of each fragment (heterosite) (C), (iii) BglII or MunI adapter at only one end of fragment (D) and, (iv) no adapter (E). A PCR reaction may then be used to obtain exponential amplification of the heterosite products (wherein different adapters are ligated to each end).

In contrast, DNA fragments with only one adapter undergo a linear amplification and are rapidly competed out in PCR, while the amplification of DNA fragments with the same adapter on each end (FIG. 1, Panels A and B) is suppressed because the self-annealing of inverted repeat adapters inhibits the binding of the PCR primers.

The use of only one set of adapters and primers diminishes competition during PCR, thereby permitting stringent reaction conditions and resulting in robustness of the method.

It should be noted that the homosite fragments (i.e., fragments that have the same type of adapter at both ends) are also valuable for detecting polymorphisms, but they should be sufficiently enriched for in a reaction, as their amplification would normally be suppressed as described. But since some homosite fragments may have originated from the annealing and ligation of adapters to restriction fragments that were created by the restriction site recognition and subsequent cleavage by two different endonucleases, such fragments may inherently contain polymorphisms of value to increasing the resolution of the typing method.

In the above-described embodiment, in principle, three different groups of PCR amplification products are generated, irrespective of which of the two selective primers is labeled (FIG. 1).

A method according to the present invention may be performed on a nucleic acid, such as a deoxyribonucleic acid or DNA, such as genomic DNA, mitochondrial DNA, plasmid DNA or cDNA or inserts or clones resulting therefrom. However, it is clear that the method according to the invention can also be based on starting material such as single-stranded (ss) or double-stranded (ds) RNA or heteroduplexes of RNA:DNA by, for example, first producing ds (c)DNA from the RNA or the heteroduplex of RNA:DNA. The DNA and/or RNA may be derived from prokaryotic organisms such as bacteria or archaea and may also be derived from viruses. Also, DNA and/or RNA to be used in a method according to the invention may be derived from eukaryotic organisms, such as eukaryotic micro-organisms like algae, fungi or yeast, or eukaryotic organisms, such as plants, like higher plants, and animals, i.e., mammals. In fact, DNA and/or RNA originating from any source or from any organism may be used in a method according to the invention. The DNA and/or RNA may be obtained from a blood sample or other suitable biological sample.

The DNA and/or RNA may be isolated using any procedure known to the person skilled in the art for the extraction of DNA and/or RNA from a sample. The method by which the DNA and/or RNA is isolated or extracted is not essential in a method according to the invention. The DNA and/or RNA may optionally be purified before being used in a method of the invention using any procedure known to the person skilled in the art for the purification of DNA and/or RNA.

A method of the invention may comprise the unspecific or random fragmentation of DNA into fragments by using unspecific methods, such as mechanical methods like sonication or French pressure cell methods, whereby the DNA is ruptured and fragmented. Also, physical methods, such as heat treatment or chemical methods for random fragmentation of DNA such as acid hydrolysis, may be employed in a method of the invention, as well as combinations thereof.

Preferably, an unspecific or random fragmentation according to the invention generates DNA fragments of between 10 to 5000 nucleotides, more preferably, 20 to 500 nucleotides. Such random fragmentation may, for example, be achieved by sonication for a predetermined period of time using a standardized or optimized intensity of sonication.

The randomly fragmented DNA, comprising fragments with both blunt ends and cohesive ends, may optionally be treated in order to provide the DNA fragments with ends that are ligatable to an adapter. Such treatment may comprise the ligation of DNA fragments to linkers that are ligatable to an adapter. Alternatively, such treatment may comprise a blunting procedure, wherein DNA fragments are treated so as to allow for the ligation of blunt-end adapters.

A method of the invention may also comprise the cleavage or cutting of DNA into fragments by using specific methods of fragmentation, such as an S1 nuclease digestion, which may specifically digest A+T-rich DNA sequences of non-coding regions of a genome, thereby leaving the specific coding fragments of the DNA. Such specific methods of fragmentation of a starting DNA may be combined with other treatments such as heat treatment. For example, when heat-treating a starting DNA to the level of partial melting, such as melting at A+T-rich regions, such single-stranded regions may then be treated by specific enzymes, thereby fragmenting the DNA in a specific manner. A specific method of fragmentation may also comprise the use of restriction endonucleases or the use of compounds, such as the camptethecin family of compounds or the enediyne antibiotics or other compounds, that preferentially bind to and cleave A+T-rich or G+C-rich DNA sequences or cause scission at minor grooves of the DNA, thereby leaving, for example, specific fragments or regions of the genome that may then be analyzed independently.

Preferably, a specific fragmentation according to the invention generates DNA fragments of between 10 to 5000 nucleotides, more preferably, 20 to 500 nucleotides.

A method of the invention preferably comprises the fragmentation of the DNA in a specific manner through cutting of the starting DNA into fragments by specific combinations of restriction endonuclease enzymes. For this, a large number of restriction endonuclease enzymes is commercially available and suitable for use in a method of the invention.

The restriction endonuclease enzymes may comprise blunt-end cutting enzymes as well as enzymes that create overhanging or recessive ends (“sticky ends”), hereinafter called cohesive ends, of varying length, depending on the specificity of the respective endonuclease used.

Suitable endonucleases comprise such enzymes that can cut between 2 to about 13 bases in a double-stranded DNA molecule, including such enzymes whose cutting sequence is located outside of its recognition sequence, but preferably cut between 4 to about 8 bases in a double-stranded DNA, more preferably, 4 to 7.

In a preferred embodiment according to the present invention, the DNA is cut with at least two endonucleases that create restriction ends that are compatible to ligation with a single adapter.

The term “compatible,” as used herein for a nucleic acid fragment end, such as a restriction end, refers to the ability of this end to align and be joined by a ligation reaction to an adapter when placed under conditions for ligation to occur, e.g., in the presence of an agent for ligation and, optionally, nucleotides. Likewise, the term “compatible,” as used herein for an adapter, refers to the ability of this adapter to align and be joined by a ligation reaction to the end of a nucleic acid fragment end, such as a restriction fragment end, when placed under conditions for ligation to occur, e.g., in the presence of an agent for ligation and, optionally, nucleotides. The term “compatible,” as used herein for two different nucleic acid fragment ends, such as two restriction fragments created by the action of two different restriction endonuclease enzymes, refers to the ability of these ends to align and be joined by a ligation reaction to a single adapter.

As can be seen in Table 1, the adapter for the MunI restriction site (as used in Example 3) is designed such that the target site for the restriction endonuclease is lost after ligation of the adapter to a MunI-derived restriction fragment. In this case, a cytosine residue was inserted in the 3′ direction of the annealing part of the overhang end of the adapter that is not base-pairing with a corresponding base in the restriction fragment. Such an adapter design is useful when performing restriction and ligation procedures in one reaction, wherein the digestion of the newly ligated fragments is undesirable. In this case, however, despite the presence of non-pairing bases, adapter ligation will readily occur between the adapter and a MunI-derived restriction fragment. It is clear that such adapters (i.e., an adapter which, after ligation to a restriction fragment, results in the loss of the restriction enzyme site) may also be used for other restriction sites.

Similarly, restriction sites created by different restriction endonucleases need not necessarily be identical to allow for a single adapter to be ligated to each of them. In fact, variations in restriction site sequence are tolerable as long as a single adapter can be ligated to such compatible restriction fragment ends. An example is the use of the restriction enzyme HgaI, with the recognition site GACGC(5/10) (SEQ ID NO:25), which may create a restriction end that is compatible to the restriction end created by the action of the enzyme MunI. As used herein, restriction fragment ends are compatible when a single adapter can be ligated to both of them.

Besides the above-mentioned at least two endonucleases that create compatible restriction ends, additional endonucleases may be used in a method according to the invention that are different from the two endonucleases that create compatible restriction ends. For such additional endonucleases, additional adapters may be provided in the case that amplification of the fragments produced from a DNA digestion with these enzymes is required.

In a preferred embodiment according to the present invention, a second set of at least two endonucleases that creates compatible ends is provided. Such a second set may be provided simultaneously or successively to a first set of at least two endonucleases that create compatible cohesive ends and may also comprise such enzymes that can cut between 2 to about 13 bases in a double-stranded DNA molecule but preferably cut between 4 to about 8 bases in a double-stranded DNA, more preferably, 4 to 7. Such an embodiment comprises the provision of a first set and a second set of restriction endonuclease enzymes that produce compatible cohesive ends within each set. As a result, only two cognate adapters, one compatible to the first set and one compatible to the second set of restriction endonuclease enzymes, are required. Alternatively, the first and second set of restriction endonuclease enzymes may each comprise a pair of restriction endonuclease enzymes, each pair consisting of two restriction endonuclease enzymes capable of cutting a starting nucleic acid in a plurality of restriction fragments with compatible cohesive ends.

The invention provides a method wherein a specific fragmentation may comprise the use of at least four different restriction endonuclease enzymes, of which at least two enzymes produce restriction fragments with ends that are compatible to a first adapter, as well as a method wherein the at least four different restriction endonuclease enzymes comprise at least a further two enzymes that produce restriction fragments with ends that are compatible to a second adapter.

Very suitable combinations of endonuclease enzymes that can be used in a method of the invention are, e.g., those provided in Table 3, which enzymes create restriction ends that are essentially identical. This table describes, for a number of endonuclease enzymes, the corresponding enzymes that create compatible cohesive ends and that can be selected to form a paired set of restriction enzymes according to the invention.

TABLE 3

Restriction endonucleases that produce compatible cohesive ends.
(Where isoschizomers exist, mostly only one member of each set is listed.
Enzymes that have degenerate recognition sequences (i.e., recognize more than
one sequence) are followed by a specific sequence in parentheses and are only
listed if a non-degenerate equivalent does not exist. The degenerate enzymes
will cleave sequences in addition to the one listed.)

| | |
|---|---|
| Endonuclease | Enzyme(s) that generate compatible cohesive ends |

| | |
|---|---|
| Acc65 I | |
| (G/GTACC) (SEQ ID NO:26) | Ban I (G/GTACC) (SEQ ID NO:26); BsiW I; BsrG I |
| Acc I | |
| (GT/CGAC) (SEQ ID NO:27) | Aci I; Acl I; BsaH I (GR/CGYC) (SEQ ID NO:28); |
| | HinP1 I; Hpa III/Msp I; Nar I; Cla I |
| | BstB I; Taq I |
| Aci I | |
| (C/CGC) (SEQ ID NO:29) | Acc I (GT/CGAC) (SEQ ID NO:27); Acl I; Cla I; |
| | BstB I; Taq I; BsaH I (GR/CGCC) (SEQ ID NO:30); |
| | HinP1 I; Nar I; Hpa II |
| Acs I | Apo I; EcoR I; Mun I; Mfe I; |
| | Tsp509 I |
| Afl III | |
| (A/CGCGT) (SEQ ID NO:31) | Asc I; BssH II |
| (A/CATGT) (SEQ ID NO:32) | BspH I; Nco I; Pci I |
| (A/CGCGT) (SEQ ID NO:31) | Mlu I |
| Age I | |
| (A/CCGGT) (SEQ ID NO:33) | Ava I (C/CCGGG) (SEQ ID NO:34); Xma I; Bsa W I; |
| | BspE I; BsrF I (A/CCGGT) (SEQ ID NO:33); SgrA I |
| | (CA/CCGGTG) (SEQ ID NO:35); NgoM IV |
| Apa I | |
| (GGGCC/C) (SEQ ID NO:36) | Ban II (GGGCC/C) (SEQ ID NO:36); Bsp1286 I |
| | (GGGCC/C) (SEQ ID NO:36) |
| ApaL I | |
| (G/TGCAC) (SEQ ID NO:37) | Sfc I (C/TGCAG) (SEQ ID NO:38) |
| Apo I | |
| (A/AATTY) (SEQ ID NO:39) | EcoR I |
| (G/AATTY) (SEQ ID NO:40) | EcoR I |
| (R/AATTY) (SEQ ID NO:12) | Mfe I; Tsp509 I |
| Asc I | |
| (GG/CGCGCC) (SEQ ID NO:41) | Afl III (A/CGCGT) (SEQ ID NO:31); Mlu I; |
| | BssH II |
| Ase I | |
| (AT/TAAT) (SEQ ID NO:42) | Bfa I; Csp6 I; Nde I; Mse I |
| Ava I | |
| (C/CCGGG) (SEQ ID NO:34) | Age I, BsaW I, BspE I, BsrF I |
| | (RICCGGY) (SEQ ID NO:43), NgoM IV, SgrA I |
| | (CR/CCGGYG) (SEQ ID NO:44), Xma I |
| (C/TCGAG) (SEQ ID NO:45) | Xho I, Sal I |
| Ava II | |
| (G/GWCC) (SEQ ID NO:46) | PpuM I (RG/GACCY) (SEQ ID NO:47); Rsr II, |
| | PpuM I (RG/GTCCY) (SEQ ID NO:48) |
| Avr II | |
| (C/CTAGG) (SEQ ID NO:49) | Nhe I, Spe I, Xba I, Sty I |
| | (C/CTAGG) (SEQ ID NO:49) |
| BamH I | |
| (G/GATCC) (SEQ ID NO:50) | Bcl I, Dpn II, Bgl II, BstY I |
| | (R/GATCY) (SEQ ID NO:l0), |
| | BstY I (G/GATCC) (SEQ ID NO:50) |
| Ban I | |
| (G/GTACC) (SEQ ID NO:26) | Acc65 I |
| (G/GCGCC) (SEQ ID NO:51) | Kas I |
| (G/GTACC) (SEQ ID NO:26) | BsiW I, BsrG I |
| Ban II | |
| (GGGCC/C) (SEQ ID NO:36) | Apa I, Bsp1286 I (GGGCC/C) (SEQ ID NO:36) |
| (GAGCT/C) (SEQ ID NO:52) | Bsp1286 I (GAGCT/C) (SEQ ID NO:52), Sac I |
| Bcl I | |
| (T/GATCA) (SEQ ID NO:9) | BamH I, BstY I (R/GATCY) (SEQ ID NO:10), Bgl II, |
| | Mbo II, Xho II (R/GATCY) (SEQ ID NO:10) |
| Bfa I | |
| (C/TAG) (SEQ ID NO:53) | Ase I, Csp6 I, Mse I, Nde I |
| Bgl II | |
| (A/GATCT) (SEQ ID NO:1) | BamH I, BstY I (R/GATCY) (SEQ ID NO:1O), Bcl I, |
| | Dpn II |
| BsaH I | |
| (GR/CGYC) (SEQ ID NO:28) | Acc I (GT/CGAC) (SEQ ID NO:27), Cla I, BstB I, Taq I |
| (GA/CGYC) (SEQ ID NO:54) | Aci I, HinPi I, Nar I |
| (GG/CGYC) (SEQ ID NO:55) | Aci I, HinPi I |
| (GG/CGYC) (SEQ ID NO:55) | Hpa II, Nar I |
| BsaW I | |
| (W/CCGGW) (SEQ ID NO:56) | Age I, BsrF I (R/CCGGY) (SEQ ID NO:43), SgrA I |
| | (CR/CCGGYG) (SEQ ID NO:44), |
| | Ava I (C/CCGGG) (SEQ ID NO:34), |
| | Xma I, BspE I, BsrF I (R/CCGGY) (SEQ ID NO:43), |
| | NgoM IV |
| BsiE I | |
| (CGAT/CG) (SEQ ID NO:57) | Pac I |
| (CGAT/CG) (SEQ ID NO:57) | Pvu I |
| (CGGC/CG) (SEQ ID NO:58) | Sac II |
| BsiHKA I | |
| (GTGCA/C) (SEQ ID NO:37) | Bsp1286 I (GTGCA/C) (SEQ ID NO:37), Bsp1286 I |
| | (GAGCA/C) (SEQ ID NO:59), |
| | Bsp1286 I (GAGCT/C) (SEQ ID NO:52), |
| | Sac I,NsiI,PstI,SbfI |
| Bsp1286 I | |
| (GGGCC/C) (SEQ ID NO:36) | Apa I, Ban II (GGGCC/C) (SEQ ID NO:36) |
| (GTGCA/C) (SEQ ID NO:37) | BsiHKA I, Nsi I, Pst I, Sbf I |
| (GAGCT/C) (SEQ ID NO:52) | Ban II (GAGCT/C) (SEQ ID NO:52), BsiHKA I, Sac I |
| (GWGCW/C) (SEQ ID NO:60) | BsiHKA I |
| BspH I | |
| (T/CATGA) (SEQ ID NO:61) | Afl III (A/CATGT) (SEQ ID NO:32), Nco I, Sty I |
| | (C/CATGG) (SEQ ID NO:62) |
| BsrF I | |
| (A/CCGGY) (SEQ ID NO:63) | Age I, BsaW I, BspE I |
| (G/CCGGY) (SEQ ID NO:64) | Age I, BsaW I, NgoM IV |
| (R/CCGGY) (SEQ ID NO:43) | Ava I, (C/CCGGG) (SEQ ID NO:34), Xma I |
| (R/CCGGY) (SEQ ID NO:43) | BsaW I, BspE I |
| (CR/CCGGYG) (SEQ ID NO:44) | SgrA I |
| BssH II | |
| (G/CGCGC) (SEQ ID NO:65) | Afl III (A/CGCGT) (SEQ ID NO:31), Mlu I, Asc I |
| BstY I | |
| (A/GATCY) (SEQ ID NO:66) | BamiH I, Bgl II |
| (G/GATCY) (SEQ ID NO:67) | BamH I |
| (R/GATCY) (SEQ ID NO:1O) | Bcl I, Dpn II |
| Eae I | |
| (Y/GGCCR) (SEQ ID NO:68) | PspOM I |
| (C/GGCCR) (SEQ ID NO:69) | Eag I, Not I |
| (T/GGCCR) (SEQ ID NO:70) | Eag I, Not I |
| Eag I | |
| (C/GGCCG) (SEQ ID NO:58) | PspOM I, Eae I (Y/GGCCR) (SEQ ID NO:68), Eae I |
| | (C/GGCCG) (SEQ ID NO:58), Not I |
| EcoR I | |
| (G/AATTC) (SEQ ID NO:11) | Apo I (G/AATTC) (SEQ ID NO:11), Apo I |
| | (R/AATTY) (SEQ ID NO:71), Mfe I, Mun I, Tsp509 I |
| MfeI | |
| (C/AATTG) (SEQ ID NO:2) | Acs I, Apo I (R/ATTTY) (SEQ ID NO:71), EcoR I, |
| | Mun I, Tsp509 I |
| Mlu I | |
| (A/CGCGT) (SEQ ID NO:31) | Afl III (A/CGCGT) (SEQ ID NO:31), Asc I, BssH II |
| Mun I | |
| (C/AATTG) (SEQ ID NO:2) | Acs I (R/AATTY) (SEQ ID NO:12), |
| | Apo I (R/AATTY) (SEQ ID NO:12), |
| | EcoR I, Mfe I, Tsp509 I |
| Nco I | |
| (C/CATGG) (SEQ ID NO:62) | Afl III (A/CATGT) (SEQ ID NO:32), BspH I, Sty I |
| | (C/CATGG) (SEQ ID NO:62), Pci I |
| Nsi I | |
| (ATGCA/T) (SEQ ID NO:72) | BsiHKA I (GTGCA/C) (SEQ ID NO:37), Bsp1286 I |
| | (GTGCA/C) (SEQ ID NO:37), Pst I, Sbf I |
| Nsp I | |
| (RCATG/Y) (SEQ ID NO:73) | Nla III, Sph I |
| Pac I | |
| (TTAAT/TAA) (SEQ ID NO:74) | BsiE I (CGAT/CG) (SEQ ID NO:57), Pvu I |
| Pci I | |
| (A/CATGT) (SEQ ID NO:32) | Afl III (A/CATGT) (SEQ ID NO:32), BspH I, Nco I |
| | Sty I (C/CTAGG) (SEQ ID NO:49) |
| PpuM I | |
| (RG/GWCCY) (SEQ ID NO:75) | Ava II, Rsr II |
| Pst I | |
| (CTGCAIG) (SEQ ID NO:38) | BsiHKA I, Bsp 1286 I (GTGCA/C) (SEQ ID NO:37), |
| | Nsi I,Sbf I |
| Pvu I | |
| (CGAT/CG) (SEQ ID NO:57) | Pac I, BsiE I (CGAT/CG) (SEQ ID NO:57) |
| Rsr II | |
| (CG/GWCCG) (SEQ ID NO:76) | Ava II, PpuM I (RG/GACCY) (SEQ ID NO:47), |
| | PpuM I (RG/GACCY) (SEQ ID NO:47), PpuM I |
| | (RG/GTCCY) (SEQ ID NO:48) |
| Sac I | |
| (GAGCT/C) (SEQ ID NO:52) | Ban II (GAGCT/C) (SEQ ID NO:52), BsiHKA I, |
| | Bsp1286 I (GAGCT/C) (SEQ ID NO:52) |
| Sbf I | |
| (CCTGCA/GG) (SEQ ID NO:77) | BsiHKA I, Bsp1286 I |
| | (GTGCA/C) (SEQ ID NO:37), Nsi I, Pst I |
| Sph I | |
| (GCATG/C) (SEQ ID NO:78) | Nla III, Nsp I |
| Sty I | |
| (C/CTAGG) (SEQ ID NO:49) | Avr II, Nhe I, Spe I, Xba I |
| (C/CATGG) (SEQ ID NO:62) | BspH I, Nco I |
| Tsp509 I | |
| (/AATT) (SEQ ID NO:79) | Apo I (R/AATTY) (SEQ ID NO:12), EcoR I, Mfe I |
| Xho II | |
| (R/GATCY) (SEQ ID NO:10) | BamH I, Bcl I, Bgl II, BstY I |
| | (R/GATCY) (SEQ ID NO:10), Dpn II |
| Xma I | |
| (C/CCGGG) (SEQ ID NO:34) | Age I, Bsa W I, BspE I, BsrF I, |
| | NgoM IV, SgrA I,Ava I |
| | (C/CCGGG) (SEQ ID NO:34) |

Legend to Single Letter IUIPAC Codes: R = A or G; Y = C or T; W = A or T.

More preferably, the digestion of DNA according to a method of the invention comprises the use of at least two different restriction endonuclease enzymes that are selected on the basis of Table 3.

In a most preferred method according to the invention, paired sets of different restriction endonuclease enzymes are chosen from the group consisting of (i) BglII, BclI, EcoRI and MunI; (ii) BglII, BclI, AcsI and MunI; (iii) BclI, XhoII, AcsI and MunI; and (iv) BglII, XhoII, EcoRI and AcsI.

The digestion of the DNA with a selection of restriction endonucleases may suitably occur in a reaction mixture comprising an aqueous solution that provides a buffered environment optimized for the activity of the respective restriction endonuclease. Such buffers are usually provided by the manufacturer along with the restriction endonuclease.

A suitable amount of DNA in a reaction mixture for performing a restriction endonuclease digestion of DNA according to the invention would be in a range of between about 1 to about 1000 ng, preferably between about 10 to about 250 ng, more preferably, between 50 and 150 ng per reaction mixture.

A suitable amount of restriction endonuclease enzyme in such a reaction mixture is generally between about 0.1 U and about 500 U of enzyme for each restriction endonuclease enzyme used. Preferably, an amount of between about 1 U and about 100 U is used, more preferably, between about 5 U and about 20 U.

A restriction endonuclease digestion of DNA according to the invention may be performed for a period of between 10 minutes and 24 hours, depending on the amount of enzyme used and the reaction temperature. Usually, optimal temperatures for the endonuclease activity of the enzyme are used with a preferred reaction period of about 1 to 10 hours. It is preferred that the reaction proceeds until digestion is essentially complete.

A restriction endonuclease digestion reaction of DNA according to the invention may be performed in one single reaction with all restriction endonuclease enzymes present. It is also possible to perform the restriction endonuclease digestion reaction of DNA in two successive reactions.

Such an embodiment is not essential, but is preferred when using two sets of restriction endonuclease enzymes that produce compatible cohesive ends or a set of two pairs of restriction endonuclease enzymes that produce compatible cohesive ends. In such a latter case, two successive double-digestion reactions are preferred. Two successive double-digestion reactions are also preferred when the restriction buffers of the different enzymes are not compatible due to, for example, different NaCl requirements. Between such successive digestion reactions, the DNA may optionally be precipitated, for example, by using a precipitating agent such as 2-propanol or ethanol.

Upon completion of the restriction endonuclease digestion reaction, the DNA fragments may be precipitated, for example, by using a precipitating agent such as 2-propanol or ethanol, and may be reconstituted in distilled water prior to use in a ligation reaction or stored for later use in such a reaction.

Suitable adapters for use in embodiments of the invention comprise double-stranded DNA adapters with ends that are compatible to ends of the DNA fragments obtained in the fragmentation of the starting DNA, such as restriction fragments, in the event that restriction endonucleases are used for fragmentation of DNA.

When using blunt-end adapters according to a method of the invention, DNA fragments with blunt ends may suitably be created from DNA fragments with cohesive ends by using ss-DNA digesting enzymes such as Exonuclease I, Mung Bean nuclease, S1 nuclease or Exonuclease VII. Alternatively, a 5′ overhanging or recessive end of a nucleic acid fragment may be filled in by using DNA polymerases such as Klenow (fragment of E. coli DNA polymerase I), T4 DNA polymerase, or Pfu polymerase which would also remove 3′ overhanging or recessive ends by the inherent 3′ to 5′ exonuclease activity, also leaving a blunt end on the nucleic acid fragment.

A method of the invention may suitably comprise the use of adapters that are blunt at one end and cohesive at the other end to allow for ligation of that adapter to the blunt-end nucleic acid fragment in a specific orientation.

As described, the term “compatible,” as used herein, refers to the ability of an adapter to align and be joined by a ligation reaction to a nucleic acid fragment, either produced by an unspecific or by a specific process for fragmentation, when placed under conditions for ligation to occur, e.g., in the presence an agent for ligation and optionally nucleotides, i.e., the adapter and the nucleic acid fragment are said to be compatible.

In the case that the nucleic acid fragment, such as a restriction fragment, and the adapter both comprise cohesive ends, the aligning will involve at least partial annealing of complementary overhanging ends. This means that the overhanging end of the adapter must be sufficiently complementary to hybridize with the respective overhanging end of the nucleic acid fragment in order to be ligated. Therefore, the sequence of the overhanging end of the adapter need not reflect the exact sequence of the respective overhanging end of the nucleic acid fragment, but may differ slightly in sequence or length.

In the present invention, an adapter and a nucleic acid fragment have compatible cohesive ends when these cohesive ends are substantially complementary. In fact, when a DNA fragmentation procedure comprises the use of at least two different restriction endonuclease enzymes that produce cohesive ends, it is sufficient for these cohesive ends to be substantially similar in order to be compatible to a single adapter.

It is a preferred embodiment of the present invention to use at least two adapters in a ligation reaction of the present invention. The at least two adapters preferably comprise a first and a second adapter, one of which is ligatable to the cohesive ends formed by at least two different restriction endonuclease enzymes used in the DNA digestion reaction.

In an even more preferred embodiment, at least two adapters are used of which a first adapter is ligatable to the cohesive ends formed by a first set of at least two restriction enzymes capable of creating compatible cohesive ends, and a second adapter is ligatable to the cohesive ends formed by a second set of at least two restriction enzymes capable of creating compatible cohesive ends.

Although more than two adapters may be used in embodiments of the present invention, the use of two adapters with a corresponding cognate amplification primer pair constitutes a most preferred embodiment of the present invention to ensure robustness of the method.

When using blunt-end adapters according to a method of the invention, the nucleic acid fragments may suitably be created by blunt-end cutting enzymes. Alternatively, cohesive ends, generated by cohesive ends-cutting endonucleases may be facilitated with blunt end by ss-DNA-digesting enzymes such as Exonuclease I, Mung Bean nuclease, S1 nuclease or Exonuclease VII. Alternatively, a 5′ overhanging or recessive end of a nucleic acid fragment may be filled-in by using DNA polymerases such as Klenow (fragment of E. coli DNA polymerase I), T4 DNA polymerase, or Pfu polymerase, which would also remove 3′ overhanging or recessive ends by the inherent 3′ to 5′ exonuclease activity, also leaving a blunt end.

When using restriction enzymes that generate blunt-end restriction fragments, a method of the invention may comprise the use of adapters that are blunt at one end and cohesive at the other end to allow for ligation of that adapter to the blunt-end restriction fragment in a specific orientation. Such adapters that are blunt at one end and cohesive on the other end may be generated by the action of restriction endonuclease on double-stranded oligodeoxy ribonucleotides or oligonucleotides. Alternatively, such adapters may be generated by the annealing of two specifically designed ss-oligodeoxy ribonucleotides that differ in length and that anneal so as to produce a ds-oligodeoxy ribonucleotide having one blunt end and one overhanging or recessive end.

Suitable adapters for use in a method according to the present invention may have a G+C content of about 50%, a length of about 3 to 50 bases, preferably, 6-30 bases, more preferably, 6-20 bases in the case of a blunt-end adapter and 18-30 bases in the case of a cohesive-end adapter and a melting temperature Tₘ of about 40-65° C., preferably 55-60° C.

Ligation of the nucleic acid fragments produced in the fragmentation procedure, such as a restriction endonuclease digestion reaction, to adapters may suitably occur in a ligase reaction mixture comprising an aqueous buffer (for example a 330 mM Tris-HCl buffer at pH 7.5) and such compounds as MgCl₂, dithiothreitol (DTT), ATP and, optionally, compounds such as polyethylene glycol (PEG). The amounts of these reagents are not essential to the invention but allow for optimal ligation reaction conditions. Suitable buffers are mostly provided by the commercial supplier of the ligase enzyme and usually comprise about 10-50 mM of MgCl₂, about 10 mM of dithiothreitol (DTT) and 0.5-10 mM of ATP.

Various ligase enzymes may be used in the ligase reaction according to the present invention. Suitable ligases are, for example, T4 DNA ligase or T7 DNA ligase. Again, the amount of ligase used in a ligase reaction in accordance with embodiments of the present invention is not essential. Suitable amounts are in the order of between 5 U to about 1000 U, preferably about 10 U to about 500 U in the case of T4 DNA ligase.

The ligase reaction mixture further comprises at least two adapters in an amount of between 1 to about 100 pM each, preferably about 10 to about 50 pM each, depending on the amount of nucleic acid fragments derived from the starting nucleic acid, or on the cutting frequency of restriction enzymes used in a specific fragmentation procedure with restriction enzymes. Usually, the adapters are present in excess over the obtainable nucleic acid fragments. The ligase reaction mixture further comprises the reconstituted nucleic acid fragments such as is produced in a restriction endonuclease digestion of o staring nucleic acid as described hereinabove in an amount of about 1 to about 1000 ng, preferably, between about 10 to about 250 ng, more preferably, between 50 and 150 ng per reaction mixture.

A ligase reaction according to the invention may be performed for a period of between 10 minutes and 24 hours, depending on the amount of enzyme used and the reaction temperature. Usually, a temperature of about 25° C. is used with a preferred reaction period of about 0.1-10 hours, more preferably about 2 hours. However, overnight ligations at 4° C. may also be used. It is preferred that the reaction proceeds until ligation is essentially complete.

A method according to the invention further comprises the amplification of adapter-ligated nucleic acid fragments. Such an amplification reaction may comprise any suitable DNA amplification reaction known to the art, such as, for example, by PCR (Mullis, 1987; U.S. Pat. No. 4,683,202), optionally in a nested, a multiplex or an asymmetric setup, or by a ligase chain reaction (Barany, 1991), a self-sustained sequence replication reaction (Guatelli et al., 1990), a transcriptional amplification system (Kwoh et al., 1989), a Q-Beta Replicase reaction (Kramer and Lizardi, 1990), a rolling circle amplification reaction (Lizardi et al., 1998), a boomerang DNA amplification reaction (BDA) (U.S. Pat. No. 5,470,724) or any other nucleic acid amplification method.

A preferred amplification reaction for use in a method according to the invention comprises a PCR amplification reaction by using Taq polymerase. Such methods are routinely employed in the art and make use of primers.

The term “primer,” as used herein, refers to an oligonucleotide, whether occurring naturally or produced synthetically, which is capable of acting as a point of initiation of synthesis when placed under conditions in which synthesis of primer extension product, which is complementary to a nucleic acid strand, is induced, i.e., in the presence of nucleotides and an agent for polymerization, such as DNA polymerase, and at suitable a temperature and pH. The primer is preferably single-stranded for maximum efficiency in amplification. Preferably, the primer is an oligodeoxy ribonucleotide. The primer must be sufficiently long to prime the synthesis of extension products in the presence of the agent for polymerization. The exact lengths of the primer will depend on many factors, including temperature and source of the primer.

The primers herein are selected to be “substantially” complementary to the different strands of each specific sequence to be amplified. This means that the primers must be sufficiently complementary to hybridize with their respective strands. Therefore, the primer sequence need not reflect the exact sequence of the template. In the present invention, the primers are substantially complementary to the adapters.

Prior to a selective amplification reaction, the nucleic acid fragments ligated to the adapters may optionally be subjected to a pre-selective amplification reaction. Such a reaction is meant to minimize artifacts and enrich for the amplification of heterosite nucleic acid fragments.

The PCR amplification primers preferably bind selectively to the complementary sequences of adapters ligated to the nucleic acid fragments. Stringent PCR conditions may be required in some applications wherein a method according to the invention is required. The stringency requirements may be most determinative to the nature of the PCR primers, i.e., their length expressed as the number of nucleotides, the most suitable annealing site on the adapter, their G+C content, their annealing temperature, etc. All these parameters may be optimized by those skilled in the art and are not essential to the present invention. A suitable length of the primers is between 8-40 nucleotides.

It is preferred that the primer anneals, especially at the 3′ end of the primer, by perfect base pairing with the DNA sequence of one of the DNA strands at the end of the adapter-ligated nucleic acid fragment. In this way, efficiency of the amplification reaction is increased. In order to amplify a subset of the adapter-ligated nucleic acid fragments, at least one primer of the pair of primers used in the amplification reaction may comprise from 1-5 selective nucleotides, preferably from 1-3 selective nucleotides at its 3′ end, that are compatible with the nucleic acid sequence. The expression “selective” herein designates the presence of nucleotides that base-pair with unknown nucleotides in the nucleic acid fragment such as those that flank the target site of the restriction endonuclease in the 3′-5′ direction of the adapter-ligated end of a restriction fragment. This measure may provide optimized discrimination by preventing the annealing of the PCR primer in the case of non-complementarity of the region such as is the case when SNPs or other nucleotide polymorphisms are present close to the restriction site.

One or both of the PCR primers may be labeled to facilitate the detection of the amplification products. Preferably, one primer is labeled. Suitable labels comprise radioactive labels, chromogenic labels, luminescent labels, phosphorescent labels, fluorescent labels such as FAM, TET, TAM, ROX, SYBR Green, Cy3, Alexa, or Texas Red, all of which are well known in the art, or other labels for detecting such tagged fragments.

Upon their amplification, the nucleic acid fragments may be detected so as to form a fingerprint of the source or organism from which the nucleic acid was extracted. A variety of different methods may be employed ranging from separation of the fragments based on size or denaturing profile on, for example, an electrophoresis gel, a capillary gel, a metaphor gel, or any other gel matrix, or by chromatography, or by an array of allelic and non-allelic markers on a DNA array.

A fingerprint is thus understood to comprise a profile of nucleic acid fragments such as a 1-dimensional banding pattern or a 2-dimensional separation pattern or an array of positive and negative spots on a support substrate.

When using sequencing gels and phospho-imaging or autoradiographic means for detection of bands, the amplification primers may accordingly be labeled to allow for such detection. A preferred embodiment comprises the use of denaturing (polyacrylamide) sequencing gels such as used in automated DNA sequencers with fluorimetric detection which will coincide with the use of at least one fluorescently labeled amplification primer, such as a FAM-labeled amplification primer and fluorescent detection.

In embodiments where a detection results in the formation of a discrete banding pattern where each band corresponds to a discrete nucleic acid fragment, the banding pattern may be scanned and analyzed by using analysis software dedicated to such tasks, such software being commercially available, resulting in a quantifiable fingerprint of the nucleic acid analyzed.

By comparing different DNA fingerprints prepared by a method according to the invention as set forth hereinabove, a method for detecting, e.g., sequence polymorphisms between one or more genomes is provided. Further, various nucleic acid fingerprints may be comparatively analyzed for the presence and/or absence of specific nucleic acid fragments.

A method for detecting DNA sequence polymorphisms according to the present invention provides the high resolution power that is required for analyzing diversity, establishing classification, or identifying taxa based on genomes from, e.g., lower level taxa such as between species, subspecies, strains, variatas, forms, cultivars or sub-populations.

Individual nucleic acid fingerprints and comparative analysis data resulting from a method according to the present invention may be stored into a computer database. In conjunction with suitable software, such a database may further be used for taxonomic or classification purposes. Also, identification of unknown organisms may be achieved by using a method for the preparation of a nucleic acid fingerprint according to the invention in combination with a database comprising a plurality, or essentially at least one, of such fingerprints. Such a database may comprise fingerprints prepared earlier from related organisms by that same method. Such a method for identification of unknown organisms may comprise comparing the fingerprint with at least one nucleic acid fingerprint prepared from at least one known organism by that same method and establishing the identity of an unknown organism on the basis of similarity of nucleic acid fingerprint from an unknown organism with at least one nucleic acid fingerprint from at least one known organism.

Use may be made of a method according to the invention for detecting sequence polymorphisms between, e.g., one or more genomes for the preparation of genetic markers. Such markers may suitably comprise SNP markers, multi-locus markers, single locus markers, dominant markers, diagnostic markers for assessing genetic differences or relatedness among individuals, populations, species, subspecies, strains, variatas, forms, cultivars, or subpopulations, phenotypic markers derived from, e.g., pedigree analysis, clonal markers to follow, e.g., recombinant gene flow and dispersal, out-crossing, introgression and hybridization, or reference markers for genetic mapping studies. Such markers may be used as molecular signatures linked to specific phenotypes and may be used to detect DNA polymorphisms associated with specific phenotypic characteristics.

It is an embodiment of the present invention to provide a kit for preparing a DNA fingerprint according to the present invention, which kit may comprise a set of restriction endonuclease enzymes and buffers for use in a method according to the present invention, a set of double-stranded DNA adapters, DNA ligase enzymes and buffers, a set of cognate primers for initiation of DNA amplification optionally labeled with a suitably detectable label, DNA polymerase enzymes and buffers, reference DNA for use in a fragmentation or a restriction digestion as a sizing standard and/or data analysis software essential to performing a method according to the invention.

In yet another embodiment, the invention provides a kit comprising at least one set of at least two different restriction endonuclease enzymes that produce restriction fragments with (two different) ends that are compatible with a single type of adapter and at least one single type of adapter.

In a preferred embodiment, the kit further comprises at least one primer that is substantially complementary to the adapter. In yet another preferred embodiment, the kit comprises at least two sets of at least two different restriction endonuclease enzymes and at least two different types of adapters and optionally comprises at least two primers of which one is substantially complementary to a first type of adapter and another primer is substantially complementary to a second type of adapter.

An example of at least one set of at least two different restriction endonuclease enzymes that produce restriction fragments with compatible cohesive ends is BglII-BclI or EcoRI-MunI. Examples of a corresponding adapter and primer can for example be found in Table 1. It is clear that a set of at least two different restriction endonuclease enzymes may be extended by another enzyme that is capable of producing a restriction fragment with the same compatible cohesive end, an example of which is the addition of XhoI to a set of BglII-BclI or the addition of AcsI/ApoI to a set of EcoRI-MunI. A set of at least two different restriction endonuclease enzymes may also be extended by the addition of an enzyme not capable of producing the same cohesive end. An example of two sets of at least two different restriction endonuclease enzymes that produce restriction fragments with compatible cohesive ends is a combination of BglII-BclI and EcoRI-MunI. Other examples are apparent to a person skilled in the art and hence no further details will be provided.

The herein disclosed genetic fingerprint method, also named Multiplex-Endonuclease Genotype Approach (“MEGA,” the terms will be used interchangeably herein) opens up possibilities with regard to diagnostics, animal breeding, forensics, parent(father)hood, pedigree and determination of the source of outbreak of a disease. Some of these possibilities are outlined hereunder in more detail.

A method according to the invention may, for example, be used in genetic mapping of quantitative trait loci (QTLs). The analysis of gene function in a trait of interest requires linkage analysis using, for example, molecular markers in a previously established genetic map. This involves identifying markers on the genetic map that co-segregate with different phenotypes of a cross, i.e., are closely linked. A number of different marker systems, such as RFLPs and AFLPs, have been used to generate genetic maps. The MEGA approach offers a finer-scale genome-wide system for the construction of a high-resolution genetic map in a variety of organisms. The procedure for such a map involves a comparison of a set each of two parental lines (P1 and P2), which are crossed to yield F1 progeny clones. DNA from the each of these clones and the parental lines are analyzed using the MEGA approach. Comparing the two parental stocks, polymorphisms are detected as the presence of a band in one stock and the absence in the other. When the progeny are examined, it can be seen that this absence/presence segregates, i.e., is linked. Such segregating markers are then used to construct a genetic map of the organism. With the MEGA approach, large numbers of markers may be analyzed simultaneously. The availability of a substantial body of genomic sequence of the organism obviates the need to undertake a cloning step as the sequences of the linked markers may be used to assemble the sequence of the genomic region of interest and from this, the identification of open reading frames (ORFs) (see Botstein & Risch, 2003, and refs. therein).

MEGA-derived polymorphisms obtained by a method according to the invention may also be used as bio-markers. The ability to identify and categorize individuals on the basis of their molecular signature profile is a powerful way of customizing a drug design process. MEGA relies on the fine-scale screening (of large numbers) of samples to identify patterns in the genetic profiles of subjects or organisms; the larger the sample population, the greater the reliability of a specific marker. To this end, one of the features of MEGA is multiplexing; that is, it has the ability to perform multiple polymorphism analyses at one time. Specific mutations in a single gene or several genes of interest may be identified, which gives an unequivocal diagnosis and affords the opportunity to attempt genotype/phenotype correlation. Identified sites of genetic variation (markers) within genes may be used to:
develop “customized medicines” that are prescribed on the basis of patients' genetic profiles. This might prevent drugs being administered to individuals in whom an adverse reaction is indicated for a specific drug.
derive biomarkers for (patient-specific) diagnostics, or used as tools for drug and/or vaccine development.
make a pre-symptomatic diagnosis of a specific disease, using specific insertion(s), deletion(s) or SNP(s) as a tag.

A specific example includes the mapping of the Apolipoprotein gene locus (Green et al., 1980), for example, as a bio-marker for coronary artery disease. The apoA, apoB and apoE genes are each amplified by PCR from a study population with ischemia or arterial damage (CAD-) and a set of control patients, and analyzed by MEGA. The results are used to determine the distribution of apoA, apoB or apoE genotype in the patient population, accorded with Hardy-Weiberg inheritance equilibrium law. The allele frequencies due to polymorphisms in restriction sites, at primer extension or resulting from insertion/deletion events between sites are determined. The genotype distributions are calculated to determine the chi values. Multivariate logistic regression analysis is used to show individuals with specific genotype, presented as a statistical chance of developing the trait of interest, and compared to individuals with the other genotypes.

Another example includes the mapping of BRCA genes (Hall et al. 1990) as markers for presymptomatic diagnosis of hereditary malignant tumors. A set of unrelated patients (preferably involving a statistical number for reliability) with clinically diagnosed cancer are screened by MEGA for mutations in BRCA genes. The genotypes are compared with those from a control population. Single nucleotide or fragment-based polymorphisms are identified and analyzed for association with malignant cancer.

Yet another example includes the mapping of chromosomal regions influencing productivity traits in livestock (for example, for breeding purposes). MEGA-derived molecular genetic markers may be used to identify chromosomal regions that contain quantitative trait loci (QTLs) that control specific traits of interest. A resource family is generated from a cross between a set of grand sires (males) and of grand dams (females). A population of F2 progeny from matings of F1 parents is derived. Phenotypic data on productivity traits are determined on the F2 population. All populations are then genotyped by MEGA, which accessed multiple independent sites within the genome. Significance thresholds are then determined by permutation tests.

A method according to the invention may also be used for analysis of (strain-)specific differences in gene expression and allelic variations in different (contrast) populations or pathogenic strains of an organism. Differential analysis of transcripts using MEGA enables the systematic analysis of alternative expression of exons, providing functional information regarding coding sequences. This involves the reverse transcription of mRNA, genotyping of the resulting cDNA by MEGA approach, and analysis of differential genotype data.

Furthermore, a method according to the invention may also be used in the population genetic structure and molecular epidemiology of populations or infectious pathogens. Using the herein described method, we studied the population genetic structure of T. brucei stocks and derived clones isolated from animal and rhodesiense sleeping sickness patients during a national sleeping sickness control program in the Mukono district, Uganda. We then performed a cladistic analysis to trace relationships and evolution, using stocks and clones recovered from geographically and temporally matched hosts, including interstrain comparisons with T. b. gambiense stocks and clones. It is clear from the obtained results (as disclosed herein) that a method according to the invention is a powerful tool in determining the population genetic structure and molecular epidemiology of populations or infectious pathogens.

The invention will now be illustrated by means of the following examples, which are in no way intended to limit the scope of the present invention.

### EXAMPLE 1

Theoretical Evaluation of the Multiplex-Endonuclease, 2-Adapter Genetic Fingerprinting Approach.

When a 6 bp-cutting endonuclease is used to digest genomic DNA, then, on average, a restriction site occurs every 4096 (4⁶) bp, assuming a random distribution of the four bases in the DNA.

When a set of four different 6 bp-cutting endonucleases that simultaneously access independent sites are used to digest the same genomic DNA, the cleavage site of each restriction enzyme would be expected to occur approximately every 1024 bp, versus approximately every 2048 bp in the case that only two 6-bp cutting enzymes are used. In practice, this will not be the case as will be discussed hereinbelow.

Assuming a random distribution of four different 6-bp-recognizing (“rare cutting”) enzymes (hypothetically A, B, C and D combination), the probability, P, of their respective digestion frequency in the genome is (P_{AB}+P_{CD}). Further assuming that the restriction enzyme cleavage sites are independent of one another and do not share nucleotides at similar positions, P is 4⁴ (4²+4²)+4⁴(4²+4²)=4⁵.

Taking, for example, the set of four “rare cutter” enzymes used in Example 3 as described hereinbelow (BglII-BclI/EcoRI-MunI), this set would generate sets of fragments with 16 possible restriction ends out of which ten different sets are potentially amplifiable (FIG. 1). The restriction enzymes were chosen such that for each pair, the cohesive ends created by one are compatible to the overhanging or recessive ends created by the other restriction enzyme. On this basis, only one pair of adapters, BglII and MunI (Table 2) was ligated to allow the amplification of the fragments using a pair of cognate primers in PCR. In this approach, BglII adapter also ligated to the overhanging or recessive ends created by BclI or XhoII, while MunI adapter also ligated to EcoRI, AcsI or ApoI sites. The short adapter ligation step ensured a substantial reduction in the amount of time required for preparing the DNA fingerprints.

With the BglII, BclI, EcoRI and MunI combination, following adapter ligation reaction, five groups of products result (FIG. 1, Panels A-E). These consist of sets of fragments with (i) BglII or MunI adapters at both ends (A, B), (ii) BglII and MunI adapters at the ends of each fragment (heterosite) (C), (iii) BglII or MunI adapter at only one end of fragment (D) and, (iv) no adapter (E). The PCR was then used to obtain exponential amplification of the heterosite products (with different adapters at each end). In contrast, DNA fragments with only one adapter undergo linear amplification and are rapidly competed out in PCR. The amplification of DNA with the same adapter at each end (FIG. 1, Panels A and B) is suppressed because self-annealing of inverted repeat adapters inhibits binding of PCR primers.

The use of only one set of adapters and primers diminishes competition during PCR, permitting robust and stringent reaction conditions. Repeat of the experiments with samples derived at two separate times from the “test” isolates gave identical AFLP profiles, with the same bands and same polymorphisms, which indicate high reproducibility of the method.

### EXAMPLE 2

Computer Predictive Modeling

For computer predictive modeling, a Monte Carlo simulation program (in QuickBasic language) was used to calculate an estimate of the occurrence of the recognition enzyme sequences. The program randomly generated a genome of adequate large size, to calculate the frequency of the recognition sequences of a set of restriction enzymes, with diverse combinations and permutations. Increasing the size of the genome and the number of repetitions was used to enhance the precision of the estimate. Also, the program is able to analyze known genome sequences. With this system, it was possible to predict the suitability of various restriction enzyme combinations in a method according to the invention.

### EXAMPLE 3

Four-Endonuclease, Two-Adapter Genetic Fingerprinting

Sets of four endonucleases in combination with two adapters were used to evaluate the value of the multiplex-endonuclease, two-adapter method as a useful tool for high-resolution DNA fingerprinting.

To identify the best set of endonucleases, we first evaluated the approach using seven “test” Trypanosoma brucei isolates belonging to the three subspecies of the parasite: T. b. brucei, T. b. gambiense and T. b. rhodesiense (Table 4). Genomic DNA, isolated essentially as previously described (Heath, 1997), was digested using restriction enzyme combinations: (i) BglII, BclI, EcoRI and MunI; (ii) BglII, BclI, AcsI and MunI; (iii) BclI, XhoII, AcsI and MunI, and; (iv) BglII, XhoII, EcoRI and AcsI, respectively. All enzymes were purchased from Roche Molecular Biochemicals (Almere, The Netherlands) or Westburg (Leusden, The Netherlands).

TABLE 4
Trypanosome isolates used

| | | Year of |
|---|---|---|
| Isolate | Source | Isolation |
| Trypanosoma brucei brucei | | |
| AnTat2/2* | Tsetse fly (Glossina morsitans); | 1970 |
| | Nigeria | |
| AnTat17/1* | Sheep; Democratic Rep. of | 1978 |
| | Congo | |
| J10* | Hyena; Zambia | 1973 |
| T. brucei gambiense | | |
| LiTat1.3* | Human; Cote de Ivoire | 1952 |
| PT16* | Human; Cote de Ivoire | 1992 |
| T. brucei rhodesiense | | |
| STIB848* | Human; Uganda | 1990 |
| AnTat12.1*⁺ | Human; Uganda | 1971 |
| T. evansi | | |
| RoTat1.2 | Water buffalo; Indonesia | 1982 |
| AnTat3.1 | Capybara; South America | 1969 |
| T. equiperdum | | |
| STIB818 | Horse; China | 1979 |
| AnTat4.1 | Unknown; South America | N/A |
| T. congolense | | |
| C49 (savannah) | Cow; Kenya | 1966 |
| Gam2 (savannah) | Cow; The Gambia | 1977 |
| IL3900 (riverine/forest) | Dog; Burkina Faso | 1980 |
| ANR3 (riverine/forest) | Fly; The Gambia | 1988 |
| K45.1 (kilifi) | Cow; Kenya | 1982 |
| WG5 (kilifi) | Goat; Kenya | 1980 |
| T. simiae | | |
| Ken2 | Fly; The Gambia | 1988 |
| TsØ2 | Bushbuck; Kenya | N/A |

*“Test” samples;
⁺Adapted sensitive to normal human serum;
N/A - data not available

Since complete digestion of DNA is an important step in the present technique, 100 ng genomic DNA was digested for four hours with 10 U of each endonuclease, essentially in two successive double-digestion reactions, with an intermediate 2-propanol precipitation step.

The digests were precipitated and reconstituted in 10 μl distilled water. Ten μl of a buffer containing 660 mM Tris HCl, 50 mM MgCl₂, 10 mM dithiothreitol, 10 mM ATP, pH 7.5, and 20 pM each of BglII and MunI adapters (Table 1) were added. One μl (400 U) of T4 DNA ligase (New England Biolabs) was added and the mixture was incubated for two hours at 25° C.

A pre-selective amplification was performed in a total volume of 20 μl containing 1 U of Taq polymerase (Roche Molecular Biochemicals, Almere, The Netherlands), 4 μl of 1:1-diluted ligation product, 2 μl of 10× PCR buffer (100 mM Tris HCl pH 9.0, 50 mM KCl, 1% triton X-100, 0.1% w/v gelatin), 2.5 mM MgCl₂, 200 tiM of each dNTP and 5 pM of each BglII and MunI primers. The reaction mixture was incubated for two minutes at 95° C., and subjected to 20 cycles of PCR (30 seconds at 95° C., 30 seconds at 56° C. and two minutes at 72° C.).

Four μl volumes of 1:20-diluted pre-selective products were used as template for a selective amplification reaction. The PCR program was essentially the same as for pre-selective amplification, except that the cycling step was followed by 30 minutes incubation at 60° C.

The final products were diluted 1:1 with TE. To a 1 μl volume of the diluted product a Genescan-500 internal lane standard (PE Applied Biosystems) was added and the mixture was resolved in a 7.3% denaturing sequencing gel using a model ABI 373A automated DNA sequencer. Gels were routinely prepared by using ABI protocols and subjected to electrophoresis for five hours.

To assess the reproducibility of the approach, two sets of genomic DNA were isolated at separate occasions from the “test” isolates and processed according to the above-mentioned protocol.

For data analysis, gel patterns were collected with GeneScan software (PE Applied Biosystems) and sample files were transferred to the GelCompar v4.1 software (Applied Maths, Kortrijk, Belgium). Band positions were inferred from the signal positions in the sample files, and used to establish the total number of bands in each lane.

By computer-assisted analysis, sample files were directly scored for presence/absence of signal peaks in the four-endonuclease or conventional approach. Gels were normalized by using the internal standard that was added to each lane. The Pearson product-moment correlation coefficient (Pearson, 1926) was used to create a similarity matrix. Clustering of the patterns was performed with the unweighed pair group method using average linkage (UPGMA) (Vauterin & Vauterin).

The approach evaluated using four-endonucleases in combination with two adapters was found to be a robust and reproducible technique capable of detecting additional amplifiable fragments to increase the number of detectable polymorphisms between genomes.

### EXAMPLE 4

Multi-Endonuclease Restriction Pattern Approach.

As an embodiment of the present invention, we describe herein an approach wherein the simultaneous use of four restriction endonucleases is able to provide greater resolution and produce more identifiable polymorphic fragments, in combination with one pair of adapters/primers for stringent PCR.

The method takes advantage of the fact that some endonucleases create cohesive ends that are compatible to the overhanging or recessive ends created by other endonucleases. On this basis, we genotyped 19 trypanosome isolates (Table 4) to the subspecies level and demonstrated the greater fingerprinting power of this approach over the conventional two-endonuclease method (FIG. 2). This approach may be useful for identifying co-dominant genetic markers or differences in closely related genomes and can easily be applied for characterization of a variety of taxa.

### EXAMPLE 5

Comparison of the Four- and Two-Endonuclease Methods

The efficiency of the conventional two-endonuclease method and a method according to the present invention using four endonuclease enzymes for generating additional polymorphisms for finer genotyping was assessed by directly comparing their fingerprint patterns using trypanosome DNA (FIG. 2).

It was shown that for all the (sub)species analyzed, samples processed using the four-endonuclease method according to the present invention revealed remarkably more restriction fragments than those analyzed with the two-enzyme method.

Moreover, extra polymorphic fragments resulted from the four-endonuclease method, indicating that additional restriction sites were indeed accessed, compared to the conventional two-endonuclease approach.

However, the increase in the number of fingerprint fragments was not linear when compared to the two-endonuclease approach with BglII and EcoRI. In principle, the pair of endonucleases, BglII (A/GATCT) (SEQ ID NO:1) and BclI (T/GATCA) (SEQ ID NO:9) when used together to digest the genome would cleave 6-bp palindromic 5′-GATC-(SEQ ID NO:80) sequences if an A or T is present at the 5′ end, respectively.

Assuming both restriction enzyme sites to be equally distributed in the genome and that all sites are cleaved, this would imply twice as much cleavage as with only one of the two enzymes, with a cutting frequency of one every 2048 (4⁶/2) bp in the genome.

In reality, this is highly unlikely due to several factors. First, the recognition sequences of the endonucleases may overlap at certain positions. In this case, depending on the restriction enzyme that cleaves first, the second enzyme may not cut since its recognition sequence would have been disrupted by the previous cleavage. Taking this into account in the simulation studies, a pair of endonucleases that share the same nucleotides at two or four positions (e.g., BglII/BclI and BglII/EcoRI), were predicted to give approximately the same mean cutting frequency, 2269 bp and 2275 bp, respectively. However, in a BglII-BclI-MunI-EcoRI digestion, a site for any of the four restriction enzymes is predicted to occur every 1501 bp.

Secondly, variations from predicted outcomes could also be due, in part, to the differential effects of methylation on enzyme sensitivity to substrate. For example, while both BglII and BclI recognition sequences completely overlap the Dam methylase site GATC, methylation does not block cleavage with BglII, but it does block cleavage with BclI, so that restriction may not occur at all BclI recognition sites. Similarly, the EcoRI recognition site (G/AATTC) (SEQ ID NO:11) is insensitive to methylation, while the cleavage of MunI (C/AATTG) (SEQ ID NO:2) site is completely blocked by Dam-methylation.

Thirdly, a common limitation of ligation-mediated restriction analyses is that, in the ligation step, two juxtaposed 5′ phosphate and 3′ hydroxyl termini in duplex DNA fragments are covalently joined by DNA ligase. As a result, the actual number of amplified fragments is less than the theoretically amplifiable fragments. Since more restriction fragments are generated in the four-endonuclease approach, this phenomenon would be expanded, and may partly account for the non-linear increase in the number of derivable amplified fragments.

Finally, liberal assumptions have been made that all four restriction enzyme recognition sequences are randomly and equally distributed in the genome, and that all restriction sites are cleaved.

### EXAMPLE 6

Trypanosome Species: intra- and inter-Species Analyses of Genetic Differences

We previously evaluated the conventional two-endonuclease method as a tool for generating useful markers for finer characterization of trypanosome isolates to subspecies level (Agbo et al., 2002). This method was limited by the fact that only marked size differences based on allelic restriction fragments from two restriction enzymes could be assessed and, as a result, only few polymorphic markers could be detected.

An important issue that arises in the context of using genetic markers for classifying individuals relates to the number of fragments and markers needed to provide adequate fingerprint or clustering. The more restriction sites are accessed in the genome, the more informative the resulting representation fragments in finding polymorphisms are.

Therefore, a minimum number of fragments (and markers) are needed for finding the underlying structural patterns of diversity in a population of interest and using this information to obtain more precise clusters of genotypes.

We have now found a unique multiplex-endonuclease method, as herein described, to expand the number of derivable fingerprint fragments, in combination with a pair of adapters and cognate primers to ensure stringency in PCR amplification.

For the inter- and intra-species analyses of trypanosome isolates (Table 1), the BglII-BclI/EcoRI-MunI combination was selected on the basis of their reproducibility, even distribution of bands along the gel and number of polymorphic bands detected (FIG. 2, Panels A-F). The intra-specific analyses of Trypanosoma brucei subspecies with four endonucleases (A) resulted in more restriction fragments and extra polymorphisms compared to the conventional two-endonuclease method (B).

Similarly, with the closely related T. evansi and T. equiperdum species (C and D), a higher number of fragments resulted with the present four-endonuclease method, but less so with the number of identifiable polymorphisms between the species. This underscores the close genetic relatedness of the two species, being probably pathotypes of the same parental strain.

The greater discriminatory power of a method according to the invention using four-endonucleases was best demonstrated in the inter-species analyses of T. congolense and T. simiae isolates, in which far more polymorphic fragments result (E) than with the two-endonuclease approach (F).

These results propose that additional sites within the genome were indeed accessed in the four-endonuclease method. When, for instance, the same region of the genome is accessed in both approaches, there is more coverage with the multi-endonuclease method so that more polymorphic restriction fragments are accessed and detected than with two or three endonucleases.

Nearly all eukaryotes contain non-coding repeat sequences that are under minimal evolutionary pressure. Therefore, since DNA polymorphisms may be concentrated at these regions, they will be highly informative for DNA fingerprinting, genetic mapping and population structure studies. It is thus possible that DNA polymorphisms can be overlooked when only a limited number of restriction enzymes have been tested with the conventional methods.

This might explain the success of the four-endonuclease method as described herein over the conventional two-endonuclease method and underline the greater inter-species genetic difference (FIG. 2, Panels E and F) when compared to differences within the members of the Trypanozoon subgenus (T. brucei, T. evansi and T. equiperdum).

Crossing human serum-resistant strains of T. brucei with susceptible strains and then assessing the phenotype of the progeny with Blood Incubation Infectivity Test (BIIT) (Rickman and Robson, 1970) has shown that human serum resistance (equivalent to human infectivity) is, at least in part, a dominant trait (Gibson and Stevens, 1999). Since restriction enzyme polymorphisms are dominant markers, the observed polymorphisms among T. brucei subspecies support the proposition (Gibson and Stevens, 1999) that the trait of human serum resistance is dominantly controlled.

Secondly, finer genetic differences, i.e., more polymorphic sites, among trypanosome (sub)species can be consistently detected by using additional sets or other combinations of endonucleases selected on the basis of the method described herein. Thereby, unique regions, such as subspecies-specific regions, may be identified to which unique markers, such as subspecies-specific markers may then be designed.

An embodiment according to the present invention wherein four-endonucleases are used has shown to give higher resolution power over the two-endonuclease method, while the use of only one pair of primers in PCR ensures stringency and robustness of the method.

Using the herein-described method, we further studied the population genetic structure of T. brucei stocks and derived clones isolated from animal and rhodesiense sleeping sickness patients during a national sleeping sickness control program in the Mukono district, Uganda. We then performed a cladistic analysis to trace relationships and evolution, using stocks and clones recovered from geographically and temporally matched hosts, including interstrain comparisons with T. b. gambiense stocks and clones.

In East Africa, certain geographically related foci are characterized by periods of long-term endemicity interspersed with short epidemic episodes. To better understand the causes of these episodes, the molecular epidemiology and population structure of T. brucei within Busoga focus have been studied (MacLeod et al., 2000; Enyaru et al., 1993; Enyaru et al., 1997; Degen et al., 1995). A recently developed PCR system based on the serum-resistance-associated (SRA) gene (De Greef et al., 1989) has been shown to be specific for the identification of T. b. rhodesiense strains (Welburn et al., 2001; Gibson et al., 2002). However, the absence of the SRA gene from other T. brucei subspecies, even in the so-called rhodesiense-like, virulent or type 2 T. b. gambiense (Gibson, 1986) underscores the need for additional genetic markers.

We undertook a population-genetic study to evaluate the population structure of parasite stocks isolated during endemic and epidemic periods within the Mukono district in Busoga focus, using the herein-described multi-locus fine genotyping marker (MEGA) system. An attractive feature of this approach is that multiple independent restriction enzyme-based polymorphisms may be genotyped in a single reaction and scored in a single lane of a gel on an automated sequencer. In this analysis, we looked for evidence of the uniqueness of circulating genotypes. Furthermore, we traced stock relationships and evolution by analyzing the extent of genetic polymorphisms among human-infective stocks on the one hand, and between human- and animal-infective stocks on the other hand, from geographically and temporally matched populations, within the same and different foci. Our data suggest that this approach offers a valuable tool for fine-scale epidemiological investigations of trypanosomosis and diseases caused by other agents.

Our results show that while there was close genetic relatedness among parasite populations from the same geographical region, microheterogeneities exist between different stocks. Data are presented that indicate that not every human sleeping sickness focus may be associated with a particular human-infective trypanosome strain responsible for long-term stability of the reference focus. We provide evidence of genetic sub-structuring among type 1 T. b. gambiense stocks, which has potentially important implications for molecular epidemiology of T. brucei.

For the above-outlined analysis, the following materials and methods were used:

Trypanosome Stocks and Clones

The trypanosome populations listed in Table 5 were originally isolated between 1990-1992 from pigs (19), cattle (5) and rhodesiense sleeping sickness patients (4) during an evaluation of the National Sleeping Sickness Control Programmme (NSSCP) in Bulutwe, the Mukono district, South-Eastern Uganda, a rhodesiense sleeping sickness endemic area. At that time, 0.7% of villagers, 33.5% of cattle and 52.8% of domestic pigs harbored trypanosome infections (Nowak et al., 1992). Human serum response properties of the parasites were evaluated earlier (Von Dobschuetz, 2002; Mangeni, unpublished data) using the Blood Incubation Infectivity Test (BIIT) (Rickman and Robson, 1980) and the Human Serum Resistance Test (HSRT) (Jenni and Brun, 1982).

TABLE 5
Origins of trypanosome populations isolated during an endemic period and
identity according to BIIT, HSRT, SRA-PCR and TgsGP19-PCR

| Stocks/Clones | | Origin | Isolation date | Host | BIIT^{a} | HSRT^{b} | SRA^{c} | TgsGP^{d} |
|---|---|---|---|---|---|---|---|---|
| 1. | SUS/BU 83/6 | Bulutwe | March 1992 | Pig | Sens° | Sens** | − | − |
| 2. | SUS/BU 83/7 | Bulutwe | April 1992 | Pig | Sens° | Sens** | − | − |
| 3. | SUS/BU 83/9 | Bulutwe | July 1992 | Pig | n.d. | Sens** | − | − |
| 4. | SUS/BU 83/9 Cl. 1 | Bulutwe | July 1992 | Pig | Subres* | Sens** | − | − |
| 5. | SUS/BU 83/9 Cl. 2 | Bulutwe | July 1992 | Pig | Sens* | Sens** | − | − |
| 6. | SUS/BU 83/9 Cl. 4 | Bulutwe | July 1992 | Pig | Subres* | Sens** | − | − |
| 7. | SUS/BU 83/9 Cl. 5 | Bulutwe | July 1992 | Pig | Subres* | Sens** | − | − |
| 8. | SUS/BU 132/2 | Bulutwe | March 1991 | Pig | n.d. | Sens** | − | − |
| 9. | SUS/BU 132/4 | Bulutwe | August 1991 | Pig | n.d. | Subres** | − | − |
| 10. | SUS/BU 139/2 | Bulutwe | March 1991 | Pig | Res* | Subres** | − | − |
| 11. | SUS/BU 169/4 | Bulutwe | August 1991 | Pig | Sens* | Subres** | − | − |
| 12. | SUS/BU 319/7 | Bulutwe | April 1992 | Pig | Subres*/sens° | Sens** | − | − |
| 13. | SUS/BU 319/7 Cl. 1 | Bulutwe | April 1992 | Pig | Sens* | Sens** | − | − |
| 14. | SUS/BU 319/7 Cl. 3 | Bulutwe | April 1992 | Pig | Subres* | Sens** | − | − |
| 15. | SUS/BU 319/9 | Bulutwe | July 1992 | Pig | Sens° | Sens** | − | − |
| 16. | SUS/BU 347/7 | Bulutwe | April 1992 | Pig | Res° | Res** | − | − |
| 17. | SUS/BU 373/7 | Bulutwe | April 1992 | Pig | Res° | Res** | − | − |
| 18. | SUS/BU 561/3 | Bulutwe | June 1991 | Pig | Subres*/sens° | Sens** | − | − |
| 19. | SUS/BU 932/7 | Bulutwe | April 1992 | Pig | Subres* | Subres** | − | − |
| 20. | BOT/BU 483/2 | Bulutwe | March 1991 | Cattle | Subres* | Sens** | − | − |
| 21. | BOT/BU 492/2 | Bulutwe | March 1991 | Cattle | n.d. | Sens** | − | − |
| 22. | BOT/BU 602/7 | Bulutwe | April 1992 | Cattle | Subres*/res° | Res** | − | − |
| 23. | BOT/BU 623/7 | Bulutwe | April 1992 | Cattle | Res° | Subres** | − | − |
| 24. | BOT/BU 1845/7 | Bulutwe | April 1992 | Cattle | Res° | Subres** | − | − |
| 25. | HOM/BU H1 | Bulutwe | November 1990 | Man | n.d. | Res** | + | − |
| 26. | HOM/BU H2 | Bulutwe | November 1990 | Man | n.d. | Res** | + | − |
| 27. | HOM/BU H5 | Bulutwe | April 1991 | Man | n.d. | Res** | + | − |
| 28. | HOM/IG 2602 | Kapyanga | February 1990 | Man | Res° | Res** | + | − |
| ^{a}Blood Incubation Infectivity Test (Rickman and Robson, 1970); °Tietjen; personal communication; *Mangeni (unpublished data); n.d.: not done. | | | | | | | | |
| ^{b}Human Serum Resistance Test (Jenni and Brun, 1982); **von Dobschuetz (2002); The HSRT was performed twice per isolate, except for BOT/BU 623/7 and BOT/BU 602/7 where it was tested three and seven times, respectively (Rickman and Robson, 1970). | | | | | | | | |
| # The isolates were defined as resistant “Res” if they showed continuous growth in the presence of human serum for at least ten days and sensitive “Sens” if they were lysed within three days. Isolates which showed non-continuous growth but | | | | | | | | |
| # remained alive in the presence of human serum for at least three days were classified as sub resistant “Subres” (Von Debschuetz, 2002). | | | | | | | | |

^{c}Serum-resistance associated (SRA) gene (present +/not present −), determined using primers defined by Gibson et al. (2002).
^{d}T. b. gambiense-specific glycoprotein (TgsGP) gene (present +/not present −), established using primers defined by Radwanska et al. (2002).

A collection of stocks and clones consisting of T. b. brucei (7), T. b. gambiense (10) and T. b. rhodesiense (8) derived during epidemic episodes from related and disparate locations (Table 6) was also analyzed to compare the genotypic properties of the various populations, separated in space and time. Parasite cloned populations were generated following limited passage of cryostabilates in laboratory rodents, according to published protocols (Hawking, 1976; Brun et al., 1981). Genomic DNA from all samples was extracted according to Heath (1997). The DNA samples were screened by PCR for the presence of the serum-resistance-associated (SRA) and T. b. gambiense-specific glycoprotein (TgsGP) genes, as described by Gibson et al. (2002) and Radwanska et al. (2002), respectively.

TABLE 6
Origin and identity of trypanosome populations
derived during epidemic outbreaks.

| Species | | Subspecies | Trypanosome stocks and clones | Origin | Isolation year | Original host |
|---|---|---|---|---|---|---|
| 1. | T. brucei | brucei | AnTat 1.8 | Uganda | 1966 | bushbuck |
| 2. | T. brucei | brucei | AnTat 2.2 | Nigeria | 1970 | tsetse |
| 3. | T. brucei | brucei | AnTat 5.2 | Gambia | 1975 | bovine |
| 4. | T. brucei | brucei | AnTat 17.1 | D.R. Congo | 1978 | sheep |
| 5. | T. brucei | brucei | Ketri 2494 ITMAS 270881 | Kenya | 1980 | tsetse |
| 6. | T. brucei | brucei | J10 ITMAS 250500A | Zambia | 1973 | hyena |
| 7. | T. brucei | brucei | TSW 196 ITMAS 300500A | Côte d'Ivoire | 1978 | pig |
| 8. | T. brucei | gambiense | AnTat 9.1 | Cameroon | 1976 | man |
| 9. | T. brucei | gambiense | LiTat 1.3 | Cote d'Ivoire | 1952 | man |
| 10. | T. brucei | gambiense | AnTat 11.17 | D.R. Congo | 1974 | man |
| 11. | T. brucei | gambiense | AnTat 22.1 | Congo/Brazzaville | 1975 | man |
| 12. | T. brucei | gambiense | JUA ITMAS 010799 | Cameroon | 1979 | man |
| 13. | T. brucei | gambiense | BAGE ITMAP 2569 | D.R. Congo | 1995 | man |
| 14. | T. brucei | gambiense | NABE ITMAP 2566 | D.R. Congo | 1995 | man |
| 15. | T. brucei | gambiense | PAKWE ITMAP 2570 | D.R. Congo | 1995 | man |
| 16. | T. brucei | gambiense | SEKA ITMAP 2568 | D.R. Congo | 1995 | man |
| 17. | T. brucei | gambiense | PT 312 | Côte d'Ivoire | 1992 | man |
| 18. | T. brucei | rhodesiense | 0404 | Rwanda | 1970 | man |
| 19. | T. brucei | rhodesiense | STIB 847 ITMAS 050399A | Uganda (Busoga) | 1990 | man |
| 20. | T. brucei | rhodesiense | STIB 848 ITMAS 190399 | Uganda (Busoga) | 1990 | man |
| 21. | T. brucei | rhodesiense | STIB 849 ITMAS 050399B | Uganda (Busoga) | 1991 | man |
| 22. | T. brucei | rhodesiense | STIB 850 ITMAS 050399C | Uganda (Busoga) | 1990 | man |
| 23. | T. brucei | rhodesiense | STIB 851 ITMAS 080399C | Uganda (Tororo) | 1990 | man |
| 24. | T. brucei | rhodesiense | STIB 882 ITMAS 080399A | Uganda | 1993 | man |
| 25. | T. brucei | rhodesiense | STIB 883 ITMAS 080399B | Uganda | 1994 | man |

Multiplex-Endonuclease Analysis

Multiplex genetic fingerprint patterns were generated for each sample according to the principle described in this patent application, which permits the simultaneous assessment of multiple independent polymorphic sites per genotyping analysis and ensures PCR stringency through the use of only one pair of adapters and primers. Briefly, 200-300 ng of genomic DNA was digested for four hours with 10 U of each restriction enzyme, in combinations BglII-BclI-EcoRI-MfeI or BglII-BclI-XhoII-EcoRI-MfeI-AcsI, respectively. The digests were precipitated and reconstituted in 10 μl distilled water. Ten μl of a buffer containing 660 mM Tris HCl, 50 mM MgCl₂, 10 mM dithiothreitol, 10 mM ATP, pH 7.5, and 20 pM of each adapter—BglII (5′-CGGACTAGAGTACACTGTC (SEQ ID NO:3); 5′-GATCGACAGTGTACTCTAGTC (SEQ ID NO:4)) and MunI (5′-AATTCCAAGAGCTCTCCAGTAC (SEQ ID NO:81); 5′-AGTACTGGAGAGCTCTTG (SEQ ID NO:82))—were added. One μl (400U) of high concentration T4 DNA ligase (New England Biolabs) was added and the mixture incubated for two hours at 25° C. Pre-selective amplification was performed in a total volume of 20 μl containing 1 U of Taq polymerase (Roche Molecular Biochemicals, Almere, The Netherlands), 4 μl of 1:1-diluted ligation product, 2 μl of 10× PCR buffer (100 mM Tris HCl pH 9.0, 50 mM KCl, 1% triton X-100, 0.1% w/v gelatin), 2.5 mM MgCl₂, 200 μM of each dNTP and 5 pM of each primer—BglII (5′-GAGTACACTGTCGATCT (SEQ ID NO:7)) and MunI (5′-GAGAGCTCTTGGAATTG (SEQ ID NO:8)). The reaction mixture was incubated for two minutes at 95° C., and subjected to 20 cycles of PCR (30 seconds at 95° C., 30 seconds at 56° C. and two minutes at 72° C.). Four μl of 1:20-diluted pre-selective products were used as template for selective primer combinations BglII-O/MfeI-A (with zero and an “A” selective nucleotide in the BglII and MfeI primers, respectively), BglII-O/MfeI-AA and BglII-0/MfeI-AT. The PCR program, electrophoresis of selective sample products, and data collection and analysis were as previously described (Agbo et al., 2002).

Phylotyping by Multiplex-Endonuclease Analysis

Genomic fingerprint patterns were generated in parallel for 13 reference populations (i.e., five T. b. brucei, six T. b. gambiense and two T. b. rhodesiense). Only fragments ranging from 35-500 bp were analyzed from which a schematic representation of the fingerprint patterns was constructed. From these profiles, identified on the basis of their intensity and individuality, a numerical matrix of observations based on the presence (1) and absence (0) of bands was built. The data were compared using Pearson correlation product moment coefficient (Pearson, 1926), which determined the proportion of mismatched bands between samples. Based on the similarity matrix, a dendrogram was generated using the unweighted pair-group method using arithmetic averages (UPGMA).

### EXAMPLE 6

Trypanosome Identity

The results of the BIIT and HSRT (summarized in Table 5) permitted an early classification of the stocks or clones derived during the endemicity survey in the Mukono district. Trypanosomes positive (resistant) in one of these tests are considered putative T. b. rhodesiense. All trypanosome DNAs were further screened by PCR for a 1.2-kb SRA fragment, scored as present or absent (+or −) (Table 5), and for a 308-bp TgsGP product (data not shown). The SRA gene fragment was amplified only from endemic trypanosome populations of human origin, which are considered genuine T. b. rhodesiense. All the samples identified as “sub-resistant” in the HSRT were negative for the SRA-specific PCR product. The specific TgsGP19 fragment was generated from only the T. b. gambiense populations.

### EXAMPLE 7

Complexity of T. brucei Populations during an Endemic Period

According to the composite BglII-BclI-EcoRI-MfeI (four-endonuclease) analysis pattern for the samples from the Mukono district (Table 5), using BglII-O/MfeI-A primer combination, the collection consisted of three phylogenetic Clusters A, B and C (FIG. 3). The pattern analysis of the control samples (data not shown), as well as human serum response trait and presence of SRA gene product, revealed that each cluster consisted of both T. b. brucei and T. b. rhodesiense. Also, the genetic relatedness and thus the distribution of the samples in the dendogram did not seem to correlate with serum response properties, and samples with the same serum response trait are not necessarily more closely related than stocks and clones with a different trait. For instance, human serum resistant SUS/BU 347/7 and SUS/BU 373/7 isolated from pig (by definition putative T. b. rhodesiense) share a genetic similarity level of 96%. On the other hand, T. b. rhodesiense HOM/BU H1 and HOM/BU H2 from different human hosts, which share 96.5% genetic similarity are 93% similar to HOM/BU H5 (Cluster B). Furthermore, HOM/IG 2602 from human in another village is distantly outplaced from other T. b. rhodesiense (to Cluster C). The similarity levels between different populations, as determined by numerical analysis of fingerprint patterns, were shown to span a continuous range of values between 79-98% (FIG. 3), with a dendogram (cophenetic) correlation of 88.4%. However, within each cluster, a highly similar genotype pattern was obtained. Overall, genotype of T. b. rhodesiense stocks from man share specific bands, which are different from other stocks (see Boxes in FIG. 4).

Since the identity of populations is highly dependent upon the resolution power of the molecular tool employed, the samples were further processed using a combination of six endonucleases and a pair of adapters. The amplified representation fragments and generated fingerprint data were similarly analyzed as described for the four-endonuclease procedure (FIG. 4). The approach consistently generated additional restriction fragments to permit for finer genome analysis, however, selection at both fragment ends seemed necessary for generating discrete fragments for further analysis. Numerical analysis of six-endonuclease fingerprint of the Mukono samples revealed two distinct clusters (FIG. 4). Cluster I comprised of a group of seven T. b. brucei stocks and clones (which share genetic similarity of more than 90%) and four T. b. rhodesiense stocks which are 77.8%, 78.5%, 80% and 85% related to other T. b. brucei stocks. Cluster II sub-divided into two subclusters comprising three T. b. rhodesiense stocks of human origin (A) and 13 T. b. brucei or T. b. rhodesiense stocks and clones (B), which share a correlation coefficient of 75.5%. Both clusters share an overall similarity that ranged between 73.5-98%, with a dendogram (cophenetic) correlation of 95% (FIG. 4). Overall, comparing the dendograms (FIGS. 3 and 4), analysis with six endonucleases revealed less genetic similarity between stocks or clones than with four enzymes.

### EXAMPLE 8

Cladistic Analysis

Genetic relatedness of the samples was evaluated on populations isolated during periods of low circulating parasitism (endemic) and upsurge in disease incidence (epidemic). Fingerprint patterns of the stocks isolated in the Mukono district, Uganda between 1990-1992 were compared to T. b. rhodesiense strains isolated during 1990-1994 epidemic episodes in the same Busoga focus. Relationships between the sets of populations inferred by numerical analysis of fingerprint data were expressed as percentage values of the Pearson correlation product moment coefficient. From the dendogram (FIG. 5), the two groups comprising endemic and epidemic stocks and clones were clearly separated and showed a percent genetic similarity coefficient of 78%. On the basis of percent genetic similarity, the dendogram was stratified into three groups. Window I represents a group of populations with 79-89% genetic similarity and considered to be “closely related.” Fingerprints in Window II comprise stocks or clones that share 90-95% genetic similarity (“highly related”), while Window III displays stocks or clones with more than 95% genetic similarity (considered “identical”). On the basis of this classification, it can be seen that T. brucei populations circulating in the Mukono district during the endemicity survey were closely related (at 82.8% for most samples, and in the range 79-82.8% for all samples). However, these are slightly different from those isolated during the epidemic within the same Busoga focus (which are 77.8% related).

Although several of the stocks isolated during the endemic survey were identical (clonal), there were major genetic differences within the Mukono T. b. rhodesiense and T. b. brucei stocks, respectively. The genotype profiles indicate that most endemic stocks were genetically separated at a similarity coefficient of 82.8%, and 79% for the stocks US/BU561/3, HOM/IG 2602 and BOT/BU 623/7 (FIG. 5). The analysis was then extended to include the genotype of T. b. brucei stocks circulating during various epidemic periods in disparate geographical regions. From the dendogram (FIG. 6), three broad groups result in which the samples isolated during the endemic survey are clustered together but share genetic similarity of only 79%. As expected, the T. b. rhodesiense stocks isolated during the 1990-1992 epidemic in Busoga were more genetically related to the T. b. rhodesiense stocks from the endemicity survey in Mukono (also within Busoga focus) than the disparate epidemic period T. b. brucei populations. A genetic correlation coefficient of 77.8% indicates that the genetic composition of T. b. brucei populations isolated during the epidemic period were remarkably different from those of other T. b. brucei isolated from the same focus during endemic periods.

When the Mukono samples were compared to all disparate stocks from epidemic episodes, the dendogram correlation showed essentially two clusters (FIG. 7). Cluster P comprises disparate T. b. brucei and T. b. gambiense populations derived outside of Busoga focus during epidemic periods, while populations from Busoga focus form Cluster Q. Each cluster is subgrouped into two subclusters differentiated on the basis of the subspecies identity of the samples. Subclusters P1 and P2 comprise disparate T. b. gambiense and T. b. brucei stocks and clones, respectively. While the samples comprising the former are genotypically not homogeneous, the latter are most heterogeneous. In addition, the T. b. gambiense stocks and clones from Cameroon, Congo and Democratic Republic of Congo are substructured into two groups (including LiTat 1.3 from Côte d'Ivoire) that share a genetic relatedness of 86.8%. This may also suggest that different genotypes were responsible for the 1995 outbreaks in the Democratic Republic (D.R) of Congo. On the other hand, Ketri 2494, isolated in Kenya (1980) from tsetse fly, grouped within Cluster Q. It is interesting that Ketri 2494 (from Kenya) is sub-clustered in Q-1, while AnTat 1.8 also a T. b. brucei isolated from Uganda (a closely related geographical area) did not fall within this group (FIG. 7). This indicates that there is close genetic relatedness of Ketri 2494 to the epidemic T. b. rhodesiense stocks from Uganda. While the samples within cluster P share a coefficient genetic relatedness of only 76.5%, the subgroups comprising cluster Q share genetic similarity of 82.5%. Populations within both clusters share only 74% genetic relatedness. As expected, intra-subspecies relatedness of the disparate T. b. brucei stocks derived during epidemic episodes was less than for those derived during the endemic survey in the Mukono district. As in previous clustering correlations (FIGS. 5 and 6), STIB 883 and now PT312 were classified as outliers in the dendogram. Their correlations to other populations were 64.8% and 59%, respectively, indicating distant genetic relatedness. In addition, it shows that the STIB 883 was completely different from other stocks circulating during the epidemic in Busoga focus and suggests that new parasite strains that upset host-parasite balance may have an important role in initiating an epidemic episode.

To summarize, we studied within- and between-stock variations using the method according to the invention to develop an understanding of the levels of variation within and between geographical foci and to describe genetic relatedness of epidemiologically defined T. brucei populations. Genome composition of the stocks seemed sufficiently stable in space and time, in agreement with the findings of Godfrey and Kilgour (1976). However, significant genetic differences exist among stocks even from within a disease focus, while T. b. brucei and T. b. rhodesiense appear to be sufficiently genetically separated to merit their retention as separate subspecies. Taken together, the data indicate that considerable genetic differences exist among T. brucei populations within the same disease focus. These differences are more evident in cladistic analysis of stocks separated in space and time of isolation. Hence, the present invention provides a powerful tool in determining the population genetic structure and molecular epidemiology of populations or infectious pathogens.

Conclusions

The present invention permits a more extensive coverage of the genome under study. The complexity of the fingerprint can be advantageously managed for finer genetic analysis by increasing the number and/or varying the choice of restriction enzyme pairs. This principle can be applied for accessing additional polymorphisms in differential display studies.

Furthermore, the present invention is also valuable in genomic DNA or c-DNA subtractive-hybridization studies where biased amplification of DNA fragments in the initial complex pool favors fragments of smaller size. As a result, the complex pool of products obtained after PCR amplification of the initial sample constitutes a “representation” of the genome, the complexity of which may be substantially less than that of the original genomic DNA. This is partially compensated for by the use of 4-bp recognizing endonucleases. However, the segment of the genome that is accessed is usually dependent upon the recognition sequence of the single endonuclease that is used. As such, the restriction enzyme recognition sequence may be biased in favor of AT- or GC-rich genomic or cDNA regions.

A number of endonuclease combinations selected on the basis of the present invention can be used to analyze additional regions of the genome, and to increase the “representation” of the generated fragments.

These features make the present invention better suited for revealing higher levels of genetic variation than with previously described methods (Lindstedt et al., 2000; Simons et al., 1997; Van der Wurff et al., 2000). For micro-restriction mapping of T. brucei subspecies, additional endonucleases in varying combinations and numbers can now be utilized to directly access more genomic regions for genetic markers that may be associated with human resistance/sensitivity.

### REFERENCES

Agbo E. C., Majiwa P. A. O, Claassen E. J. H. M. and Roos M. H. (2001). Measure of molecular diversity within the Trypanosoma brucei subspecies Trypanosoma brucei brucei and Trypanosoma brucei gambiense as revealed by genotypic characterization. Exp. Parasitol., 99, 123-131.
Agbo E. C., Majiwa P. A. O., Claassen E. H. J. M. and Pas M. F. W. (2002). Molecular variation of Trypanosoma brucei subspecies as revealed by AFLP fingerprinting. Parasitol. 124, 349-358.
Barany, F. (1991). Genetic disease detection and DNA amplification using cloned thermostable ligase. Proc. Natl. Acad. Sci. USA 88(1), 189-193.
Botstein D., Risch N. (2003). Discovering genotypes underlying human phenotypes: past successes for mendelian disease, future approaches for complex disease. Nat. Genet. 33 Suppl: 228-237.
Brun R., Jenni L., Schönenberger M., Schell K-F. (1981). In vitro cultivation of bloodstream forms of Trypanosoma brucei, T. rhodesiense and T. gambiense. J. Protozool. 28, 470-479.
De Greef C., Imbrechts G., Matthyssens G., Van Meirvenne N., Hamers R. (1989). A gene expressed only in serum-resistant variants of Trypanosome brucei rhodesiense. Mol. Biochem. Parasitol. 36, 169-176.
Degen R., Pospichal H., Enyaru J. C. K., Jenni L. (1995). Sexual compatibility among Trypanosoma brucei isolates from an epidemic area in Southeastern Uganda. Parasitol. Res. 8, 253-257.
Enyaru J. C. K., Matovu, E., Odiit M., Okedi L. A., Rwendeire A. J. J., Stevens J. R. (1993). Isoenzyme comparison of Trypanozoon isolates from two sleeping sickness areas of South-eastern Uganda. Acta Tropica 55, 97-115.
Enyaru K. C. K., Matovu E., Odiit M., Okedi L. A., Rwendeire A. J. J., Stevens J. R. (1997). Genetic diversity in Trypanosoma (Trypanozoon) brucei isolates from mainland and Lake Victoria island populations in South-eastern Uganda: Epidemiological and control implications. Ann. Trop. Med. Parasitol. 91, 107-113.
Gibson W. C and Stevens J. (1999). Genetic exchange in Trypanosomatidae. Advs. in Parasitol., 43, 1-45.
Gibson W. C., Marshall T. F. de C. and Godfrey D. G. (1980). Numerical analysis of enzyme polymorphism: a new approach to epidemiology and taxonomy of trypanosomes of the subgenus Trypanozoon. Adv. in Parasitol., 18, 175-246.
Gibson W., Backhouse T., Griffiths A. (2002). The human serum-resistance associated gene is ubiquitous and conserved in Trypanosoma brucei rhodesiense throughout East Africa. Infec. Genet. Evol. 1, 207-214.
Gibson W. C. (1986). Will the real Trypanosoma b. gambiense please stand up. Parasitol. Today 2, 255-257.
Godfrey D. G., Kilgour V. (1976). Enzyme electrophoresis in characterizing the causative organism of gambian trypanosomiasis. Trans. R. Soc. Trop. Med. Hyg. 70, 219-224.
Green P. H. R., Glickman R. M., Riley J. W., Quinet E. (1980). Human apolipoprotein. A-IVFintestinal origin and distribution in plasma. J. Clin. Invest. 65:911-919.
Guatelli J. C., Whitfield K. M., Kwoh D. Y., Barringer K. J., Richman D. D. and Gingeras T. R. (1990). Isothermal, in vitro amplification of nucleic acids by a multienzyme reaction modeled after retroviral replication. Proc. Natl. Acad. Sci. USA 87(5), 1874-1878.
Hall J. M., Lee M. K., Newman B., Morrow J. E., Anderson L. A., Huey B., King M. C. (1990). Linkage of early-onset familial breast cancer to chromosome 17q21. Science 250: 1684-1689.
Hawking F. (1973). The differentiation of Trypanosoma rhodesiense from T. brucei by means of human serum. Transactions of the Royal Soc. of Trop. Med. and Hygiene, 67, 517-527.
Hawking F. (1977). The resistance to human plasma of Trypanosoma brucei, T. rhodesiense and T. gambiense. In: Analysis of the composition of trypanosome strains. Transactions of the Royal Soc. of Trop. Med. and Hygiene, 70, 504-512.
Hawking F. (1976). The resistance to human plasma of Trypanosoma brucei, Trypanosoma rhodesiense and Trypanosoma gambiense. In: Analysis of the composition of trypanosome strains. Trans. R. Soc. Trop. Med. Hyg. 70, 504-512.
Heath S. (1997). Molecular Techniques in Analytical Parasitology. In Analytical Parasitology (ed. Rogan, M. T.), pp 67-68.
Hide G. (1999). History of sleeping sickness in East Africa. Clin. Microbiol. Rev. 12, 112-125.
Hide G., Welburn S. C., Tait A., Maudlin I. (1994). Epidemiological relationships of Trypanosoma brucei stocks from South East Uganda: evidence for different populations structures in human infective and non-human infective isolates. Parasitol. 109, 95-111.
Jenni L., Brun R. (1982). An in vitro test for human serum resistance of Trypanosoma (Trypanozoon) brucei. Acta Tropica 39, 281-284.
Kassai K. (1988). Standardized nomenclature of animal parasitic disease (SNOAPAD). Vet. Parasitol., 29, 299-326.
Kramer F. R., Lizardi P. M. (1990). Ampliflable hybridization probes. Ann. Biol. Clin. (Paris). 48(6), 409-11.
Kwoh D. Y., Davis G. R., Whitfield K. M., Chappelle H. L., DiMichele L. J., Gingeras T. R. (1989). Transcription-based amplification system and detection of amplified human immunodeficiency virus type 1 with a bead-based sandwich hybridization format. Proc. Natl. Acad. Sci. USA 86(4), 1173-1177.
Lindstedt B.-A., Heir E., Vardund T. and Kapperd G. (2000). A variation of the amplified fragment length polymorphism (AFLP) technique using three restriction endonucleases, and assessment of the enzyme combination BglII-MfeI for AFLP analysis of Salmonella enterica subsp. enterica isolates. FEMS Microbiol. Letts., 189,19-24.
Lizardi P. M., Huang X., Zhu Z., Bray-Ward P., Thomas D. C. and Ward D. C. (1998). Mutation detection and single-molecule counting using isothermal rolling-circle amplification. Nature Genetics 19:225-232.
MacLeod A., Turner C. M., Tait A. (1999). A high level of mixed Trypanosoma brucei infections in tsetse flies detected by three hypervariable minisatellites. Mol. Biochem. Parasitol. 102, 237-248.
MacLeod A., Turner C. M., Tait A. (2001a). The detection of geographical substructuring of Trypanosoma brucei populations by the analysis of minisatellite polymorphisms. Parasitol. 123, 475-482.
MacLeod A., Tweedie A., Welburn S. C., Maudlin I., Turner C. M., Tait A. (2000). Minisatellite marker analysis of Trypanosoma brucei: reconciliation of clonal, panmictic, and epidemic population genetic structures. Proc. Natl. Acad. Sci. USA 97, 13442-13447.
MacLeod A., Welburn S., Maudlin I., Turner C. M., Tait A. (2001b). Evidence for multiple origins of human infectivity in Trypanosoma brucei revealed by minisatellite variant repeat mapping. J. Mol. Evol. 52, 290-301.
Mueller U. G. and Wolfenbarger L. L. (1999). AFLP genotyping and fingerprinting—a review. Trends in Ecol. & Evol., 14, 389-394.
Mullis K. B. and Faloona F. A. (1987). Specific synthesis of DNA in vitro via a polymerase-catalyzed chain reaction. Methods Enzymol., 155, 335-50.
Nowak F., Kakaire D., Tietjen U., Hoffmann L., Katabazi B., Mahlitz D. (1992). Glossina f. fuscipes as a vector of human and animal trypanosomiasis in South-eastern Uganda. Determination of seasonal fly density, host preference and infection rates. Zbl. Bakt. Hyg. 325, 60-61.
Pearson K. (1926). On the coefficient of racial likeness. Biometrika, 18, 105-117.
Radwanska M., Claes F., Magez S., Magnus E., Perez-Morga A., Pays E., Buscher P. (2002). Novel primer sequences for polymerase chain reaction-based detection of Trypanosoma brucei gambiense. Am. J. Trop. Med. Hyg. 67, 289-295.
Rickman L. R and Robson J. (1970). The testing of proven Trypanosoma brucei and T. rhodesiense strains by the blood incubation infectivity test. Bulletin of the World Health Organization 42, 911-916.
Simons G., van der Lee T., Diergaarde P., van Daelen R., Groenendijk J., Frijters A., Buschges R., Hollricher K., Topsch S., Schulze-Lefert P., Salamini F., Zabeau M. and Vos P. (1997). AFLP-based fine mapping of the Mlo gene to a 30-kb DNA segment of the barley genome. Genomics, 44, 61-70.
Van der Wurff A. W. G., Chan Y. L., van Straalen N. M. and Schouten J. (2000). TE-AFLP: combining rapidity and robustness in DNA fingerprinting. Nucleic Acids Res., 28, e105.
Vauterin L. A. and Vauterin P. (1992). Computer-aided objective comparison of electrophoresis patterns for grouping and identification of microorganisms. European Microbiol. J., 1, 37-41.
Von Dobschuetz S. (2002). Molekularbiologische Untersuchungen zur Identifizierung potentiell humaninfektiöser Trypanosoma (Trypanozoon) brucei—Isolate aus Süd-Ost Uganda. Doctoral Dissertation, Journal-Nr. 2577, Freie Universität Berlin, p. 205.
Vos P., Hogers M., Bleeker M., Reijans M., van de Lee T., Homes M., Frijters A., Pot J., Peleman J., Kuiper M. and Zabeau M. (1995). AFLP: a new technique for DNA fingerprinting. Nucleic Acids Res., 23, 4407-4414.
Welburn S. C., Picozzi K., Fevre E. M., Coleman P. G., Odiit M., Carrington M., Maudlin. I. (2001). Identification of human-infective trypanosomes in animal reservoir of sleeping sickness in Uganda by means of serum-resistance-associated (SRA) gene. Lancet 358, 2017-2019.

1. A process for producing a nucleic acid fingerprint, the process comprising:
fragmenting a starting nucleic acid, thus producing a plurality of adapter ligatable nucleic acid fragments having ends that are compatible to at least one adapter;
performing a ligation reaction between said ends of said plurality of adapter ligatable nucleic acid fragments and the at least one adapter, thus producing adapter-ligated nucleic acid fragments;
amplifying said adapter-ligated nucleic acid fragments with at least one amplification primer that is essentially complementary to a nucleotide sequence of said at least one adapter, thus producing amplified adapter-ligated nucleic acid fragments; and
generating a nucleic acid fingerprint from said amplified adapter-ligated nucleic acid fragments.

2. The process according to claim 1, wherein said plurality of adapter ligatable nucleic acid fragments are obtained by specific fragmentation of said starting nucleic acid.

3. The process according to claim 2, wherein said specific fragmentation comprises digesting said starting nucleic acid with at least two different restriction endonuclease enzymes, thus producing restriction fragments having different ends that are compatible to a single adapter.

4. The process according to claim 2, wherein said specific fragmentation comprises digesting said starting nucleic acid with at least three different restriction endonuclease enzymes, wherein at least two of said at least three different restriction endonuclease enzymes produce restriction fragments having ends that are compatible to a single adapter.

5. The process according to claim 2, wherein said specific fragmentation comprises digesting said starting nucleic acid with at least four different restriction endonuclease enzymes, wherein at least two of said at least four different restriction endonuclease enzymes produce restriction fragments having ends that are compatible to a first adapter.

6. The process according to claim 5, wherein said at least four different restriction endonuclease enzymes comprise at least two enzymes that produce restriction fragments having ends that are compatible to a second adapter.

7. The process according to claim 3, wherein said ends of said restriction fragments comprise cohesive ends.

8. The process according to claim 3, wherein said restriction endonuclease enzymes are 4 to 8 bp-cutting restriction endonuclease enzymes.

9. The process according to claim 3, wherein said restriction endonuclease enzymes that produce compatible ends are selected from the group of enzymes listed in Table 3.

10. The process according to claim 5, wherein said at least four different restriction endonuclease enzymes are selected from the group consisting of: BglII, BclI, EcoRI and MunI; BglII, BclI, AcsI and MunI; BclI, XhoII, AcsI and MunI; and BglII, XhoII, EcoRI and AcsI.

11. The process according to claim 10, wherein said first adapter is a BglII adapter and said second adapter is a MunI adapter.

12. The process according to claim 1, wherein performing the ligation reaction between said ends of said plurality of adapter ligatable nucleic acid fragments and said at least one adapter comprises forming heterosite nucleic acid fragments.

13. The process according to claim 1, wherein amplifying said adapter-ligated nucleic acid fragments with said at least one amplification primer comprises using at least one primer having selective nucleotides.

14. The process according to claim 1, wherein generating the nucleic acid fingerprint from said amplified adapter-ligated nucleic acid fragments comprises electrophoresing said amplified adapter-ligated nucleic acid fragments.

15. The process according to claim 14, further comprising visualizing said nucleic acid fingerprint with fluorimetry, autoradiography, phospho-imaging, or other methods of visualizing nucleic acid fingerprints.

16. The process according to claim 1, wherein said starting nucleic acid is genomic DNA.

17. A method for detecting sequence polymorphisms between one or more genomes, the method comprising:
producing at least one nucleic acid fingerprint from one or more genomes with a process, the process comprising:
fragmenting the one or more genomes, thus producing a plurality of adapter ligatable nucleic acid fragments having ends that are compatible to at least one adapter;
performing a ligation reaction between said ends of said plurality of adapter ligatable nucleic acid fragments and the at least one adapter thus producing adapter-ligated nucleic acid fragments;
amplifying said adapter-ligated nucleic acid fragments with at least one amplification primer that is essentially complementary to a nucleotide sequence of said at least one adapter, thus producing amplified adapter-ligated nucleic acid fragments; and
generating at least one nucleic acid fingerprint from said amplified adapter-ligated nucleic acid fragments; and
comparing the at least one nucleic acid fingerprint for the presence of, the absence of, or differences between said amplified adapter-ligated nucleic acid fragments to determine the presence of sequence polymorphisms.

18. The method according to claim 17, wherein the one or more genomes are genomes from lower level taxa.

19. The method according to claim 18, wherein said lower level taxa comprises species, subspecies, strains, variatas, forms, cultivars or sub-populations.

20. The method according to claim 17, wherein the one or more genomes are genomes from parasites.

21. A method for the identification of DNA markers linked to a specific phenotype, a phenotypic characteristic, a genetic trait or any combination thereof, the method comprising:
detecting sequence polymorphisms with the method according to claim 17 between said one or more genomes originating from organisms which exhibit differences in the specific phenotype, the phenotypic characteristic, the genetic trait or any combination thereof; and
correlating said polymorphisms to said differences.

22. The method according to claim 21, wherein said DNA markers are selected from the group consisting of: SNP markers, multi-locus markers, single locus markers, dominant markers, diagnostic markers, phenotypic markers, clonal markers, reference markers, and any combination thereof.

23. A method for identifying an unknown organism, the method comprising:
producing a nucleic acid fingerprint from said unknown organism with a process, the process comprising:
fragmenting a starting nucleic acid thus producing a plurality of adapter ligatable nucleic acid fragments having ends that are compatible to at least one adapter;
performing a ligation reaction between said ends of said plurality of adapter ligatable nucleic acid fragments and the at least one adapter, thus producing adapter-ligated nucleic acid fragments;
amplifying said adapter-ligated nucleic acid fragments with at least one amplification primer that is essentially complementary to a nucleotide sequence of said at least one adapter, thus producing amplified adapter-ligated nucleic acid fragments; and
generating a nucleic acid fingerprint from said amplified adapter-ligated nucleic acid fragments;
comparing said nucleic acid fingerprint with at least one known nucleic acid fingerprint produced from at least one known organism with said process; and
establishing an identity of said unknown organism on the basis of similarity of said nucleic acid fingerprint from said unknown organism with said at least one known nucleic acid fingerprint from said at least one known organism.

24. A kit for performing the process according to claim 1, comprising an element selected from the group of elements consisting of restriction endonuclease enzymes, buffers, double-stranded DNA adapters, DNA ligase enzymes, cognate amplification primers, DNA polymerase enzymes, reference DNA and data analysis software, and any combination thereof.

25. The kit of claim 24, wherein the cognate amplification primers are labeled with a detectable label.

26. A kit comprising:
at least one set of at least two different restriction endonuclease enzymes capable of producing restriction fragments having ends that are compatible with a single type of adapter; and
at least said one single type of adapter.

27. The kit of claim 26, further comprising at least one primer that is essentially complementary to said adapter.

28. The process according to claim 1, wherein the at least one adapter comprises a first and a second set adapter and the at least one amplification primer comprises two primers having selective nucleotides.
